SEQUENCE LISTING (85 sequences)

SEQ ID NO: 1 - protein, 27 aa, Homo sapiens
YDGRGDSVVYGLKKKAARGRRAARGRR

SEQ ID NO: 2 - protein, 30 aa, Homo sapiens
PYAGRGDSVVYGLGGGPGAARGRRAARGRR

SEQ ID NO: 3 - protein, 29 aa, Homo sapiens
PYDGRGDSVVYGLRKKKAARGRRAARGRR

SEQ ID NO: 4 - protein, 29 aa, Homo sapiens
ATSLPPHSSQSPGGGPPAARGRRAARGRR

SEQ ID NO: 5 - protein, 30 aa, Homo sapiens
AARGRRAARGRRKKKAPYAGRGDSVVYGLR

SEQ ID NO: 6 - protein, 29 aa, Homo sapiens
RRGRAARRGRAAKKKRLGYVVSDGRGDYP

SEQ ID NO: 7 - protein, 29 aa, Homo sapiens
RLGYVVSDGRGDYPKKKRRGRAARRGRAA

SEQ ID NO: 8 - protein, 27 aa, Homo sapiens
ATSLPPHSSQSPKKKAARGRRAARGRR

SEQ ID NO: 9 - protein, 27 aa, Homo sapiens
PSQSSHPPLSTAKKKRRGRAARRGRAA

SEQ ID NO: 10 - protein, 27 aa, Homo sapiens
RRGRAARRGRAAKKKPSQSSHPPLSTA

SEQ ID NO: 11 - protein, 30 aa, Homo sapiens
RRGRAAKKKRRGRAAKKKPSQSSHPPLSTA

SEQ ID NO: 12 - protein, 33 aa, Homo sapiens
RRGRAARRGRAARRGRAAKKKPSQSSHPPLSTA

SEQ ID NO: 13 - protein, 32 aa, Homo sapiens
RRGRAAKKKRRGRAAKKKRLGYVVSDGRGDYP

SEQ ID NO: 14 - protein, 29 aa, Homo sapiens
PSQSSHPPLSTAPPGGGPSQSSHPPLSTA

SEQ ID NO: 15 - protein, 29 aa, Homo sapiens
ATSLPPHSSQSPPPGGGPSQSSHPPLSTA

SEQ ID NO: 16 - protein, 14 aa, Homo sapiens
RLGYVVSDGRGDYP

SEQ ID NO: 17 - protein, 15 aa, Homo sapiens
RRGRAARRGRAAKKK

SEQ ID NO: 18 - protein, 33 aa, Homo sapiens
RAAKKRARAAKKRARAAKKRLGYVVSDGRGDYP

SEQ ID NO: 19 - protein, 33 aa, Homo sapiens
KRAAKKAAKRAKKAAKKAARLGYVVSDGRGDYP

SEQ ID NO: 20 - protein, 34 aa, Homo sapiens
RKKAARARKKAARARKKAARRLGYVVSDGRGDYP

SEQ ID NO: 21 - protein, 18 aa, Homo sapiens
RRGRAAKKKRRGRAAKKK

SEQ ID NO: 22 - protein, 21 aa, Homo sapiens
RKRAARARKRAARARKRAARR

SEQ ID NO: 23 - protein, 34 aa, Homo sapiens
RKRAARARKRAARARKRAARRLGYVVSDGRGDYP

SEQ ID NO: 24 - protein, 19 aa, Homo sapiens
RAAKKRARAAKKRARAAKK

SEQ ID NO: 25 - protein, 19 aa, Homo sapiens
KRAAKKAAKRAKKAAKKAA

SEQ ID NO: 26 - protein, 20 aa, Homo sapiens
RKKAARARKKAARARKKAAR

SEQ ID NO: 27 - protein, 19 aa, Homo sapiens
RRGRAAKKKRRGRAAKKKR

SEQ ID NO: 28 - protein, 20 aa, Homo sapiens
RRGRARRGRARRGRARRGKK

SEQ ID NO: 29 - protein, 12 aa, Homo sapiens
ATSLPPHSSQSP

SEQ ID NO: 30 - protein, 12 aa, Homo sapiens
PSQSSHPPLSTA

SEQ ID NO: 31 - protein, 12 aa, Homo sapiens
ATSLPPHSSQSP

SEQ ID NO: 32 - protein, 7 aa, Homo sapiens
SLPAHAR

SEQ ID NO: 33 - protein, 7 aa, Homo sapiens
HSSLQTP

SEQ ID NO: 34 - protein, 7 aa, Homo sapiens
YHSSFQA

SEQ ID NO: 35 - protein, 15 aa, Homo sapiens
ATSLPPHECQSPLRC

SEQ ID NO: 36 - protein, 25 aa, Homo sapiens
ALAQSHVLPPHSSQSPPLCARPPHA

SEQ ID NO: 37 - protein, 7 aa, Homo sapiens
ATWLPPR

SEQ ID NO: 38 - protein, 7 aa, Homo sapiens
TPHNTVS

SEQ ID NO: 39 - protein, 14 aa, Homo sapiens
CATSLPPHSSQSPC

SEQ ID NO: 40 - protein, 18 aa, Homo sapiens
CGPATSLPPHSSQSPGPC

SEQ ID NO: 41 - protein, 8 aa, Homo sapiens
KPSSPPEE

SEQ ID NO: 42 - protein, 5 aa, Homo sapiens
  misc_feature (3)..(4): Xaa can be any naturally occurring amino acid
LWXXX

SEQ ID NO: 43 - protein, 7 aa, Homo sapiens
  misc_feature (1)..(1): Xaa can be any naturally occurring amino acid
XFXXYLW

SEQ ID NO: 44 - protein, 10 aa, Homo sapiens
CRRHWGFEFC

SEQ ID NO: 45 - protein, 10 aa, Homo sapiens
CTTHWGFTLC

SEQ ID NO: 46 - protein, 12 aa, Homo sapiens
WHSDMEWWYLLG

SEQ ID NO: 47 - protein, 16 aa, Homo sapiens
NGYEIEWYSWVTHGMY

SEQ ID NO: 48 - protein, 12 aa, Homo sapiens
HTMYYHHYQHHL

SEQ ID NO: 49 - protein, 8 aa, Homo sapiens
SHWSPWSS

SEQ ID NO: 50 - protein, 14 aa, Homo sapiens
KRFKQDGGWSHWSP

SEQ ID NO: 51 - protein, 18 aa, Homo sapiens
KRFKQDGGWSHWSPWSSC

SEQ ID NO: 52 - protein, 19 aa, Homo sapiens
SPWSSCSVTCGDGVITRIR

SEQ ID NO: 53 - protein, 6 aa, Homo sapiens
VYMSPF

SEQ ID NO: 54 - protein, 7 aa, Homo sapiens
MQLPLAT

SEQ ID NO: 55 - protein, 5 aa, Homo sapiens
CNGRC

SEQ ID NO: 56 - protein, 9 aa, Homo sapiens
CLSSRLDAC

SEQ ID NO: 57 - protein, 7 aa, Homo sapiens
SMSIARL

SEQ ID NO: 58 - protein, 15 aa, Homo sapiens
HGRFILPWWYAFSPS

SEQ ID NO: 59 - protein, 14 aa, Homo sapiens
YYAWHWYAWSPKSV

SEQ ID NO: 60 - protein, 24 aa, Homo sapiens
NGRKICLDLQAPLYKKIIKKLLES

SEQ ID NO: 61 - protein, 19 aa, Homo sapiens
QRFTTMPFLFCNVNDVCNF

SEQ ID NO: 62 - protein, 15 aa, Homo sapiens
KNNQKSEPLIGRKKT

SEQ ID NO: 63 - protein, 12 aa, Homo sapiens
PLAEIDGIELTY

SEQ ID NO: 64 - protein, 16 aa, Homo sapiens
GHGLGHGHEQQHGLGH

SEQ ID NO: 65 - protein, 7 aa, Homo sapiens
ATWLPPR

SEQ ID NO: 66 - protein, 8 aa, Homo sapiens
NSAVQLEN

SEQ ID NO: 67 - protein, 7 aa, Artificial Sequence
  Structurally modified peptide derived from Thrombospondin-1
GVITRIR

SEQ ID NO: 68 - protein, 18 aa, Homo sapiens
  MISC_FEATURE (1)..(18): D-amino acids
ARRGRAARRGRAARRGRA

SEQ ID NO: 69 - protein, 20 aa, Homo sapiens
  MISC_FEATURE (1)..(20): D-amino acids
RRGRARRGRARRGRARRGKK

SEQ ID NO: 70 - protein, 34 aa, Homo sapiens; identical to SEQ ID NO: 23
  MISC_FEATURE (1)..(34): D-amino acids

SEQ ID NO: 71 - protein, 21 aa, Homo sapiens
  MISC_FEATURE (1)..(21): D-amino acids
RKGRRKGRRKGRRKGRRKGKK

SEQ ID NO: 72 - protein, 31 aa, Homo sapiens
  MISC_FEATURE (1)..(31): D- amino acids
RRGRRRGRRRGRRRGKKRLGYVVSDGRGDYP

SEQ ID NO: 73 - protein, 17 aa, Homo sapiens
RRGRRRGRRRGRRRGKK

SEQ ID NO: 74 - protein, 17 aa, Homo sapiens
RRQRRRGRRRQRRRGKK

SEQ ID NO: 75 - protein, 32 aa, Homo sapiens; identical to SEQ ID NO: 13
  MISC_FEATURE (1)..(32): D- amino acids

SEQ ID NO: 76 - protein, 17 aa, Homo sapiens
  MISC_FEATURE (1)..(2): D-amino acids
RRARRRARRRARRRAKK

SEQ ID NO: 77 - protein, 29 aa, Homo sapiens
  MISC_FEATURE (1)..(29): D-amino acids
RRGRRRGRRRGRRRGKKPSQSSHPPLSTA

SEQ ID NO: 78 - protein, 30 aa, Homo sapiens
RRGRRRGRRRGRRRGKKRLGYVVSDGRGDY

SEQ ID NO: 79 - protein, 31 aa, Homo sapiens
RRGRRRGRRRGRRRGKKRLGYVVSDGRAAYP

SEQ ID NO: 80 - protein, 31 aa, Homo sapiens
RRGRRRGRRRGRRRGKKRLGYVVSAARGDYP

SEQ ID NO: 81 - protein, 31 aa, Homo sapiens
RRGRRRGRRRGRRRGKKRLGYVVSAARAAYP

SEQ ID NO: 82 - protein, 31 aa, Homo sapiens
RRGRRRGRRRGRRRGKKRAGAVVSDGRGDYP

SEQ ID NO: 83 - protein, 30 aa, Homo sapiens
RRGRRRGRRRGRRRGKKRAGAVVSAARAAP

SEQ ID NO: 84 - protein, 7 aa, Unknown
  heparin binding domain
XXXXXXX

SEQ ID NO: 85 - protein, 6 aa, Unknown
  heparin binding domain
XXXXXX

(19) United States
(12) Patent Application Publication
(10) Pub. No.: US 2006/0172941 A1
(43) Pub. Date: Aug. 3, 2006

(54) Anti-angiogenic peptides and methods of use thereof

(76) Inventors: Luca Rastelli, New Haven, CT (US); Judith Landin, New Haven, CT (US); Uriel Malyankar, New Haven, CT (US); Richard Kitson, New Haven, CT (US); Melissa Corso, New Haven, CT (US); Kenneth Brunson, New Haven, CT (US)

Correspondence Address: COOLEY GODWARD LLP, THE BROWN BUILDING - 875 15TH STREET, NW, SUITE 800, WASHINGTON, DC, 20005-2221, US

(21) Appl. No.: 11/327,849

(22) Filed: Jan. 9, 2006

Related U.S. Application Data
(63) Continuation-in-part of application No. PCT/US05/36959, filed on Oct. 14, 2005.
(60) Provisional application No. 60/618,273, filed on Oct. 14, 2004.

(51) Int. Cl. A61K 39/00; C07K 14/475

(52) U.S. Cl. 514/12; 530/350

(57) ABSTRACT

Anti-angiogenic peptides that inhibit activation or proliferation of endothelial cells are disclosed. Such peptides may be used to inhibit VEGF binding to the VEGFR2 receptor (also known as the kinase domain receptor or KDR) and bFGF binding to its receptor. Such peptides may also be used to inhibit, VEGF, bFGF, or integrin activation of endothelial cells in angiogenesis-associated diseases such as cancer, leukemia, multiple myeloma, inflammatory diseases, eye diseases and skin disorders.

### CROSS-REFERENCE TO RELATED APPLICATIONS

This application claims benefit of priority to U.S. provisional application 60/618,273, which is herein incorporated by reference in its entirety.

### FIELD OF INVENTION

This application relates to the identification and design of therapeutic peptides for treatment and characterization of angiogenesis-related diseases and tumorigenesis-related diseases, particularly anti-angiogenic peptides that block binding of vascular endothelial growth factor (VEGF) to its receptor, VEGFR2, also known as the kinase domain receptor or kinase insert domain-containing receptor (KDR). While VEGF acting via KDR is a major angiogenic factor, several other ligand-receptor interactions are implicated during angiogenesis. This invention discloses a series of bifunctional peptides where the VEGF receptor binding peptide is linked to peptides that inhibit angiogenesis by binding or interfering with other angiogenic receptors and pathways.

### BACKGROUND OF INVENTION

Angiogenesis is the process by which new blood vessels form by developing from pre-existing vessels. This multi-step process involves signaling to endothelial cells, which results in (1) dissolution of the membrane of the originating vessel, (2) migration and proliferation of the endothelial cells, and (3) formation of a new vascular tube by the migrating cells (Alberts et al., 1994, Molecular Biology of the Cell. Garland Publishing, Inc., New York, N.Y. 1294 pp.). While this process is employed by the body in beneficial physiological events such as wound healing and myocardial infarction repair, it is also exploited by unwanted cells such as tumor cells, and in undesirable conditions such as atherosclerosis, inflammatory conditions such as dermatitis, psoriasis, and rheumatoid arthritis, as well as eye diseases such as diabetic retinopathy and macular degeneration.

Angiogenesis is required for the growth and metastasis of solid tumors. Studies have confirmed that in the absence of angiogenesis, tumors rarely have the ability to develop beyond a few millimeters in diameter (Isayeva et al., 2004, Int. J. Oncol. 25(2):335-43). Angiogenesis is also necessary for metastasis formation by facilitating the entry of tumor cells into the blood circulation and providing new blood vessels that supply nutrients and oxygen for tumor growth at the metastatic site (Takeda et al., 2002, Ann Surg. Oncol. 9(7):610-16).

Endothelial cells are also active participants in chronic inflammatory diseases, in which they express various cytokines, cytokine receptors and proteases that are involved in angiogenesis, proliferation and tissue degradation. For example, during rheumatoid arthritis, endothelial cells become activated and express adhesion molecules and chemokines, leading to leukocyte migration from the blood into the tissue. Endothelial cell permeability increases, leading to oedema formation and swelling of the joints (Middleton et al., 2004, Arthritis Res. Ther. 6(2):60-72).

Abnormal neovascularization is also seen in various eye diseases, where it results in hemorrhage and functional disorder of the eye, contributing to the loss of vision associated with such diseases as retinopathy of prematurity, diabetic retinopathy, retinal vein occlusion, and age-related macular degeneration (Yoshida et al., 1999, Histol Histopathol. 14(4):1287-94). These conditions are the leading causes of blindness among infants, those of working age and the elderly (Aiello, 1997, Ophthalmic Res. 29(5):354-62).

Understanding angiogenesis is also of crucial importance for the treatment of skin diseases, as it is a key contributor to pathologic dermatological processes such as psoriasis, warts, cutaneous malignancy, decubitus ulcers, stasis ulcers, pyogenic granulomas, hemangiomas, Kaposi's sarcoma, and possibly Spitz nevus, hypertrophic scars, and keloids (Arbiser, 1996, J. Am. Acad. Dermatol. 34(3):486-97). Thus, recent developments in the understanding of angiogenesis will likely lead to advances in the treatment of skin cancer, psoriasis and other skin diseases, and more rapid healing of wounds.

Multiple myeloma is the second most common blood cancer, representing approximately one percent of all cancers and two percent of all cancer deaths. Multiple myeloma still represents a major unmet medical need, and there is a need to develop compounds that can treat this disease with a good safety profile. Understanding angiogenesis is crucial for the treatment of this disease.

Vascular endothelial growth factor (VEGF) is a particularly potent angiogenic factor that acts as an endothelial cell-specific mitogen during angiogenesis (Binetruy-Tourniere et al., 2000, EMBO J. 19(7): 1525-33). VEGF has been implicated in promoting solid tumor growth and metastasis by stimulating tumor-associated angiogenesis (Lu et al., 2003, J. Biol. Chem. 278(44): 43496-43507). VEGF has been found in the synovial fluid and serum of patients with rheumatoid arthritis (RA), and its expression is correlated with disease severity (Clavel et al., 2003, Joint Bone Spine. 70(5): 321-6). VEGF has also been implicated as a major mediator of intraocular neovascularization and permeability. Transgenic mice overexpressing VEGF demonstrate clinical intraretinal and subretinal neovascularization, and form leaky intraocular blood vessels detectable by angiography, demonstrating their similarity to human disease (Miller, 1997, Am. J. Pathol. 151(1):13-23).

Given the involvement of pathogenic angiogenesis in such a wide variety of disorders and diseases, inhibition of angiogenesis, and particularly of VEGF signaling, is a desirable therapeutic goal. VEGF acts through two high affinity tyrosine kinase receptors, VEGFR1 (or fms-like tyrosine kinase, Flt-1), and VEGFR2 (also known as kinase domain receptor or kinase insert domain-containing receptor, KDR). Although VEGFR1 binds VEGF with a 50-fold higher affinity than KDR, KDR appears to be the major transducer of VEGF angiogenic effects, i.e., mitogenicity, chemotaxis and induction of tube formation (Binetruy-Tourniere et al., supra). Inhibition of KDR-mediated signal transduction by VEGF, therefore, represents an excellent approach for anti-angiogenic intervention.

In this regard, inhibition of angiogenesis and tumor inhibition has been achieved by using agents that either interrupt VEGF/KDR interaction and/or block the KDR signal transduction pathway, including antibodies to VEGF (Kim et al., 1993, Nature 362, 841-844; Kanai et al., 1998, J. Cancer 77, 933-936; Margolin et al., 2001, J. Clin. Oncol. 19, 851-856); antibodies to KDR (Lu et al., 2003, supra; Zhu et al., 1998, Cancer Res. 58, 3209-3214; Zhu et al. 2003, Leukemia 17, 604-61 1; Prewett et al., 1999, Cancer Res. 59, 5209-5218); anti-VEGF immunotoxins (Olson et al., 1997, Int. J. Cancer 73, 865-870); ribozymes (Pavco et al., 2000, Clin. Cancer Res. 6, 2094-2103); soluble receptors (Holash et al., 2002, Proc. Natl. Acad. Sci. USA 99, 11393-11398; Clavel et al. supra); tyrosine kinase inhibitors (Fong et al., 1999, Cancer Res. 59, 99-106; Wood et al., 2000, Cancer Res. 60, 2178-2189; Grosios et al., 2004, Inflamm Res. 53(4):133-42); antisense mediated VEGF suppression (Forster et al., 2004, Cancer Lett. 20;212(1):95-103); and RNA interference (Takei et al., 2004, Cancer Res. 64(10):3365-70; Reich et al., 2003, Mol Vis. 9:210-6). Peptides that block binding of VEGF to KDR have also been described, and were shown to inhibit VEGF-induced angiogenesis in a rabbit corneal model (Binetruy-Tourniere et al., 2000, EMBO J. 19(7): 1525-33). Still, given the wide variety of patients that stand to benefit from the development of effective anti-angiogenic treatments, there remains a need for the further identification and characterization of novel anti-angiogenic drug compounds.

Recently, Genentech introduced to the market a recombinant humanized anti-VEGF monoclonal antibody, Avastin (bevacizumab). This antibody has shown efficacy in the treatment of colon cancer, and is being tested on other tumor cell types. Cost analysis suggests that treatment with this antibody could add from $42,800 to $55,000 per patient to the cost of care for advanced colorectal cancer, or more than $1.5 billion annually in the United States. Thus, there is a need for alternative drugs such as small peptides that are less expensive to manufacture and may be used therapeutically at a much lower cost.

Although VEGF activation of KDR is a major angiogenic pathway, several other ligand-receptor interactions are implicated in angiogenesis. The involvement of these other ligand-receptor interactions in VEGF mediated tumor-induced angiogenesis may explain why, for instance, Avastin is very effective at treating colon cancer but is much less effective at treating breast cancer. In breast cancer, it is believed that genetic variability and instability of tumor cells leads to the expression of multiple growth factors. As the Avastin example illustrates, there is a need for alternative drugs such as the multifunctional peptides of the present invention which are capable of blocking multiple ligand-receptor interactions.

### SUMMARY OF INVENTION

The present inventors have identified using mini peptide display technology novel anti-angiogenic and anti-tumorigenic peptides that not only block or reduce VEGF-induced stimulation of endothelial cell activation or proliferation but also target pathways and receptors that play a role in angiogenesis. For example, some of the peptides are competitive inhibitors for integrin activation. Others affect interactions of endothelial cells with matrix components. Still others affect the binding of growth factors, including but not limited to VEGF, fibroblast growth factors (FGF), heparin-binding epidermal growth factor (HBEGF), and hepatocyte growth factor (HGF), to their receptors by binding the heparin sulfate moieties presented by endothelial cells. Finally, some of the peptides are competitive inhibitors of enzymes that are required for migration and invasion through the basement membrane like the MMPs and uPaR complex.

In one embodiment of the present invention, the peptides demonstrate a significantly lower IC50 and/or greater affinity for heparin when compared to previously known peptides. In addition, the fusion peptides composed of two or more anti-angiogenic peptides demonstrate a synergistic effect, i.e. the activity of the fusion peptide is qualitatively and quantitatively better than the sum of the individual peptides. Accordingly, the peptides of the invention are useful for the treatment of angiogenesis-related diseases, including the treatment of tumors and neoplasias, inflammatory diseases such as rheumatoid arthritis and psoriasis, vascular disorders including atherosclerosis, vascular restenosis, arteriovenous malformations and vascular adhesion pathologies, and eye diseases including diabetic retinopathy and macular degeneration.

The invention provides anti-angiogenic fusion peptides comprising a first peptide linked to a second peptide through an optional linker peptide. The fusion peptides have inhibitory activity against one or more receptors involved in different angiogenic pathways. The fusion peptides are represented by the general formula (I):
(A)m−L−(B)n (I)

wherein L is an optional linker peptide comprising about 0-10 amino acids;

wherein each A and B are independently peptides comprising about 1-about 35 amino acids;

wherein m and n are independently integers from about 1-3.

In the fusion peptides of the invention, at least one of A and B comprises an amino acid sequence that binds one or more cell surface components such as VEGF receptors, integrin receptors, heparin sulfate proteoglycan, and FGF receptors and enzymes like the MMPs and uPaR.

### BRIEF DESCRIPTION OF THE DRAWINGS

FIG. 1 shows a phylogenetic tree generated by clustalW using Vector NTI, which compares the relationship between the peptides identified using mini peptide display technology and the peptides disclosed in Binetruy-Tournaire R, Demangel C, Malavaud B, Vassy R, Rouyre S, Kraemer M, Plouet J, Derbin C, Perret G, Mazie J C. EMBO J. 2000 Apr. 3; 19(7):1525-33, and Lu D, Shen J, Vil M D, Zhang H, Jimenez X, Bohlen P, Witte L, Zhu Z. J Biol Chem. 2003 Oct. 31; 278(44):43496-507.

FIG. 2 shows a homology alignment between the peptides: EmboK4 (SEQ ID No. 32), EmboK5 (SEQ ID No. 33) and EmboV4 (SEQ ID No. 34) from the paper by Binetruy-Tournaire et al., the two peptides 1A11 and 2D5 (which have the same sequence (SEQ ID No. 35) and therefore will be considered as one) from the paper by Lu et al., and the clone K3 (SEQ ID No. 36) obtained by mini peptide display technology.

FIG. 3 shows a further homology alignment including K3 and the two of the peptides disclosed by Binetruy-Tournaire et al., EmboV1 (SEQ ID No. 37) and EmboK3 (SEQ ID No. 38).

FIG. 4 is a graph showing VEGF-mediated survival/proliferation of bovine retinal endothelial cells (BRE cells) in the presence of peptide ST100,038 (SEQ ID NO.: 29).

FIG. 5 is a graph showing VEGF-mediated survival/proliferation of bovine retinal endothelial cells (BRE cells) in the presence of peptides ST100,059 (SEQ ID NO.: 30) and ST100,068 (SEQ ID NO.: 10).

FIG. 6 is a graph showing the inhibition of bFGF-mediated survival/proliferation of human umbilical endothelial cells in the presence of peptides ST100,068 (SEQ ID NO.: 10), ST100,072 (SEQ ID NO.: 11), and ST100,073 (SEQ ID NO.: 12).

FIG. 7 is a graph showing VEGF binding inhibition by peptides ST100,032 (SEQ ID NO.: 1) and ST100,033 (SEQ ID NO.: 29), where both peptides at a concentration of 30 μM completely abolished VEGF binding.

FIG. 8 is a graph showing VEGF or bFGF-mediated survival/proliferation of human dermal microvasculature endothelial cells in the presence of peptide ST100,061 (SEQ ID NO.: 3).

FIG. 9 is a graph comparing peptide ST100,064 (SEQ ID NO.: 6) with peptide ST100,061 (SEQ ID NO.: 3) in the inhibition of bFGF-mediated survival/proliferation of human umbilical endothelial cells.

FIG. 10 is a graph showing the inhibition of bFGF-mediated survival/proliferation of human umbilical endothelial cells in the presence of several peptides.

FIG. 11 is a graph showing the inhibition of proliferation of mouse leukemia L1210 cells in the presence of ST100,077 (SEQ ID NO.: 16), ST100,078 (SEQ ID NO.: 17) and ST100,064 (SEQ ID NO.: 6).

FIG. 12 is a graph showing inhibition of growth of melanoma B16 tumor xenograft in vivo treated with 20 mg/kg daily IP of ST100,059 (SEQ ID NO.: 30), ST100,061 (SEQ ID NO.: 3) and ST100,062 (SEQ ID NO.: 4) as compared to untreated controls.

FIG. 13 is a graph showing inhibition of growth of melanoma B16 tumor implanted subcutaneously treated in vivo with 20 mg/kg daily IP and 40 mg/kg daily IP of ST100,068 (SEQ ID NO.: 10).

FIG. 14 is a graph showing inhibition of growth melanoma B15 tumor implanted subcutaneously treated in vivo with 20 mg/kg daily IP of ST100,073 (SEQ ID NO.: 12).

FIG. 15a is a graph showing inhibition of growth of mouse leukemia L1210 IV treated in vivo with various amounts of miniproteins administered IP. FIG. 15b is a graph showing inhibition of growth of mouse leukemia L1210 IV treated in vivo with various amounts of miniproteins administered IV.

FIG. 16 is a graph showing inhibition of growth of RPMI-8226 human multiple myeloma xenographs implanted subcutaneously and treated with 25 mg/kg daily of ST100,064 (SEQ ID NO.: 6) and 100 mg/kg daily of ST100,059 (SEQ ID NO.: 30) administered IP.

FIG. 17 is a graph showing the inhibition of bFGF-mediated survival/proliferation of human umbilical endothelial cells in the presence of several peptides.

### DETAILED DESCRIPTION OF THE INVENTION

Peptides

The present inventors have identified novel anti-angiogenic peptides. The term “anti-angiogenic” means that the peptides of the invention block, inhibit or reduce the process of angiogenesis, or the process by which new blood vessels form by developing from pre-existing vessels. Such peptides can block angiogenesis by blocking or reducing any of the steps involved in angiogenesis, including the steps of (1) dissolution of the membrane of the originating vessel, (2) migration and proliferation of the endothelial cells, and (3) formation of the new vascular tube by the migrating cells.

In particular, the peptides of the invention block, inhibit or reduce VEGF-induced stimulation of endothelial cell activation or proliferation, as may be detected or measured using any one or more of the assays described herein or in the available literature. For instance, the ability of the disclosed peptides to inhibit or reduce VEGF-induced stimulation may be measured by incubating the disclosed peptides in the presence of VEGF and monitoring any reduction in the proliferation or survival of bovine retinal endothelial cells (BRE) or human umbilical vein endothelial cells (HUVEC) as described herein. Other measures of endothelial cell stimulation may also be used, including detecting the affect of the peptides on the expression of one or more anti-apoptotic proteins such as Bcl-2 and Al (see Gerber et al., 1998, J. Biol. Chem. 273(21): 133313-16), or the affect of the peptides on the phosphorylation or dephosphorylation of VEGF signal transducing proteins such as Akt (see Gerber et al., 1998, 273(46): 30336-43).

The peptides of the invention also block, inhibit or reduce VEGF binding to the KDR receptor, as may be detected or measured using the disclosed mini peptide technology, or any known competitive or non-competitive KDR receptor binding assay. In this regard, labeled minicells or any other cell expressing a peptide of the invention may be used to detect or measure binding of the disclosed peptides to the KDR receptor. The present invention also encompasses labeled peptide derivatives of any of the peptides disclosed herein, wherein the peptide is conjugated or complexed to a detectable label such as a radioactive, fluorescent, luminescent, proteogenic, immunogenic or any other suitable molecule.

The term “peptide” as used in the present invention is equivalent with the term “polypeptide” and refers to a molecule comprising a sequence of at least six amino acids, but does not refer to polypeptide sequences of whole, native or naturally occurring proteins. Thus, the peptides of the invention have at least six amino acids and preferably not more than about 100, 75, 50, 40, 30, 25, 20 or 15 amino acids. Most preferred peptides of the invention will have at least about six amino acids.

The term “miniprotein” as used in the present invention is a protein containing two or more domains. Generally, miniproteins are synthetic peptides.

Based on homology alignment of the peptides identified using mini peptide display technology with KDR blocking peptides of the prior art, the inventors identified a consensus sequence of LPPHSS (residues 4 to 9 of SEQ ID No. 4) that provides the core sequence for a novel family of peptides having substantially improved anti-angiogenic properties. This core consensus sequence was further expanded by homology alignment to include at least one or more of the N-terminal amino acids ATS, and/or at least one or more of the C-terminal amino acids QSP, creating expanded consensus sequences of ATSLPPHSS (residues 1 to 9 of SEQ ID No. 4), LPPHSSQSP (residues 9 to 12 of SEQ ID No. 4) and ATSLPPHSSQSP (residues 1 to 12 of SEQ ID No.4). See U.S. provisional application 60/599,059, which is herein incorporated by reference in its entirety.

Peptides comprising the amino acid sequence of SEQ ID No.4 in particular have been shown to demonstrate a significantly lower IC50 of about 40 versus about 200 micromolar when compared to previously known peptides. Accordingly, peptides of the present invention demonstrate the functional attributes of anti-angiogenic activity, and may further block or reduce VEGF binding to KDR at a concentration of less than about 200 micromolar, more preferably at a concentration less than about 175, 150, 125, 100 or 75 micromolar, and most preferably at a concentration less than about 50 micromolar.

Data from the literature indicates that transforming linear peptides into constrained cyclic peptides often increases their activity. The present invention contains bifunctional cyclic peptides based on the sequences C-ATSLPPHSSQSP-C (SEQ ID No. 39) and C-GPATSLPPHSSQSPGP-C (SEQ ID No. 40), where intramolecular bonds are generated between the terminal cysteines.

In addition, while VEGF acting via KDR is a major angiogenic factor, several other ligand-receptor interactions play a role during angiogenesis, especially tumor-induced angiogenesis (see Eccles S A, 2004, Int J Dev Biol. 48: 583-98.). These other ligand-receptor interactions are also targeted by the bifunctional peptides of the present invention.

For instance, heparan sulfates (HS) presented on the cellular membrane by proteoglycans have been implicated in the regulation of cell growth and differentiation by modulating the activity of growth factors. Various growth factors such as fibroblast growth factors (FGFs), vascular endothelial growth factor, heparin-binding epidermal growth factor, and hepatocyte growth factor (HGF), bind to HS and heparin and form tight complexes. HS facilitate the binding of growth factors to their receptors with at least two mechanisms. In the first, HS and heparin bind to growth factors in a multivalent manner and induce oligomerization of the growth factors, which is responsible for growth factor receptor dimerization, activation, and signaling. In the second, HS and heparin promote the activity of growth factors by simultaneously binding to regions on both the growth factor and its receptor. As such, a target for anti-angiogenesis activity can be the co-receptor activity of HS.

Accordingly, the present invention comprises bifunctional peptides comprising heparin and HS binding domains. The heparin binding domain follows two general consensus sequences: bbbxxbx (SEQ ID No. 84) and bbxbxx (SEQ ID No. 85)(where b is any basic amino acid (arginine or lysine) and x is any amino acid that favors helical structure including but not limited to alanine (A) or glycine (G)). The domain may be repeated. For example, the concensus sequence can be represented as (bbbxxbx)ₙ (SEQ ID No. 84) or (bbxbxx)ₙ (SEQ ID No. 85), wherein n is any number including but not limited to 1, 2, 3, 4, and 5. In general bbbxxbx (SEQ ID No. 84) has stronger binding activity than bbxbxx (SEQ ID No. 85) because the higher the number of basic residues was found to correlate with stronger heparin binding activity.

In one embodiment, among others, the heparin binding bifunctional peptide of the present invention can comprise any one of the following heparin binding sequences:

| | |
|---|---|
| RAAKKRARAAKKRARAAKK | (SEQ ID NO.:24) |
| KRAAKKAAKRAKKAAKKAA | (SEQ ID NO.:25) |
| RKKAARARKKAARARKKAAR | (SEQ ID NO.:26) |
| RRGRAAKKKRRGRAAKKKR | (SEQ ID NO.:27) |
| RRGRARRGRARRGRARRGKK | (SEQ ID NO.:28) |

In addition, two growth factor families activate an initiating pathways in angiogenesis: the vascular endothelial growth factors and fibroblast growth factors (FGF). Both of them require co-receptors, neuropilin-1 for VEGF (Klagsbrun et al., 2002, Adv. Exp. Med. Biol. 515: 33-48) and heparin sulfate proteoglycan (glypicans and syndecan) for FGF and some VEGF isoforms (Ornitz and Itoh, 2001, Genome Biol. 2(3): 3005(1-12) and Iozzo and San Antonio, 2001, J. Clin. Invest. 108(3): 349-355). In addition, endothelial cell migration, proliferation of new lumen during angiogenesis require coordinated interactions with the extracellular matrix (ECM). Several ECM components act via the integrin family of receptors that are the major attachment and migration receptors (Jin H., 2004, Br. J. Cancer. 90(3): 561-5.). Finally, several enzymes are required for migration and invasion through the basement membrane like the MMPs and uPaR complex.

TABLE 1 is a list of other small peptides described in the

literature that interact with receptors or co-receptors in
angiogenesis, and may form the basis of bifunctional
antiangiogenic peptides as described in the present invention.

| | | | |
|---|---|---|---|
| Peptide | sequence | Target | Publication |

| | | | |
|---|---|---|---|
| A6 | KPSSPPEE | uPAR inhibitor | Guo et al., 2000, FASEB J. |
| | (SEQ ID No. 41) | | 14(10):1400-10. |
| | LWxxAr | uPAR inhibitor | Goodson et al. PNAS 91 |
| | (Ar = Y, W, F, H) | | 7129. |
| | (SEQ ID No. 42) | | |
| | Xfxxylw | | |
| | (SEQ ID No. 43) | | |
| | CRRHWGFEFC | mmp9 inhibitor | Koivunen et al. Net. Biot |
| | (SEQ ID No. 44) | | 17 768. |
| | CTTHWGFTLC | mmp2 inhibitor | Koivunen et al. Net. Biot |
| | (SEQ ID No. 45) | | 17 768. |
| Peptide F56 | WHSDMEWWYL | bind flt-1 | An et al., 2004, Int J |
| | LG | | Cancer. 111(2):165-73. |
| | (SEQ ID No. 46) | | |
| SP5.2 | NGYEIEWYSWV | bind flt-1 | El-Mousawi et al., 2003, J |
| | THGMY | | Biol Chem. 278(47): |
| | (SEQ ID No. 47) | | 46681-91. |
| K237 | HTMYYHHYQH | bind kdr | Hetian et al., 2002, J Biol |
| | HL | | Chem. 277(45):43137-42. |
| | (SEQ ID No. 48) | | |
| | shwspwss | bind to heparin | Guo et al., 1992, J Biol |
| | (SEQ ID No. 49) | | Chem. 267(27):19349-55. |
| | krfkqdggwshwsp | bind to heparin | Guo et al., 1992, J Biol |
| | (SEQ ID No. 50) | | Chem. 267(27):19349-55. |
| TSP 599 | KRFKQDGGWS | bind to heparin | Guo et al., 1992, J Biol |
| | HWSPWSSC | | Chem. 267(27):19349-55. |
| | (SEQ ID No. 51) | | |
| TSp 616 | SPWSSCSVTCG | anti-angiogenic | Guo et al., 1992, J Biol |
| | DGVITRIR | | Chem. 267(27):19349-55. |
| | (SEQ ID No. 52) | | |
| | VYMSPF | FGF receptor | Fan et al. IUBMB life |
| | (SEQ ID No. 53), | | 54:67; Maruta et al. Cancer |
| | MQLPLAT | | Gene Therapy 9:543. |
| | (SEQ ID No. 54) | | |
| | CNGRC | endothelium | Arap et al., 2002, Science. |
| | (SEQ ID No. 55) | binder via | 99:1527 |
| | | aminopeptidase | |
| | | N/CD13 | |
| | CLSSRLDAC | brain | Arap et al., 2002, Science. |
| | (SEQ ID No. 56) | endothelium | 99:1528. |
| | SMSIARL | prostate | Arap et al., 2002, Science. |
| | (SEQ ID No. 57) | endothelium | 99:1529. |
| | HGRFILPWWYA | Thomsen- | Peletskaya et al. J. Mol. |
| | FSPS | Friedenreich | Biol. 270 374. |
| | (SEQ ID No. 58) | antigen | |
| | YYAWHWYAWS | | |
| | PKSV | | |
| | (SEQ ID No. 59) | | |
| | NGRKICLDLQA | FGF-2 | Hagerdon et al., 2001, The |
| | PLYKKIIKKLLE | | FASEB Journal. |
| | S (HEPARIN | | 15:550-552. |
| | BINDING) | | |
| | (SEQ ID No. 60) | | |
| Endostatin | | | Chillemi et al. |
| fragments | | | |
| Tumstatin | QRFTTMPFLFCN | alphavbeta3 | Maeshima et al. JBC 276 |
| peptides | VNDVCNF | integrin | 31959. |
| | (SEQ ID No. 61) | | |
| Fibronectin | KNNQKSEPLIGR | heparin binding | Haugen et al., 1990, J Cell |
| | KKT- | | Biol. 111:2733-45. |
| | (SEQ ID No. 62) | | |
| Tenascin | PLAEIDGIELTY | alpha9beta1 | Schneider, 1998, FEBS |
| fragment | (SEQ ID No. 63) | integrin | Lett. 429(3):269-73. |
| Kininogen | GHGLGHGHEQQ | Neutrophil | Colman et al., 2000, |
| fragment | HGLGH | binding site | Blood. 95:543-550. |
| 440-455 | (SEQ ID No. 64) | | |
| | ATWLPPR | KDR | Binetruy-Tournaire et al., |
| | (SEQ ID No. 65) | | 2000, EMBO J. |
| | | | 19:1525-33. |
| Prothrombin | NSAVQLEN | Prothrombinase | Kim et al., 2002, Thromb |
| kringle-2 | (SEQ ID No. 66) | | Res. 106:81-7. |
| region | | | |
| Thrombospondin- | NacetylGVDITRI | TSP-1 | Haviv et al. 2005, J. |
| 1 | Rnethylmaleimide | | Med. Chem. 48(8) |
| | (SEQ ID No. 67) | | 2838-2846. |
| Decorin | 26 amino acid | Decorin | Sulochana et al. 2005, J. |
| Leucine | peptide leucine- | | Biol Chem. 280(30), |
| rich repeat | rich repeat 5 | | 27935-48 |
| region | | | |
| Prothrombin | NSAVQLEN | Prothrombinase | Kim et al. Thromb Res. |
| kringle-2 | (SEQ ID No. 66) | | 2002 Apr 1; 106(1):81-7. |
| region | | | |
| 2nd | | MCP-1-CCR2 | Kim et al. 2005, FEBS |
| extracellular | | interaction | Lett. 579(7), 1597-601. |
| loop of CCR2 | | | |
| and CCR3 | | | |

The present invention provides peptides with anti-angiogenic activity. These peptides target pathways and receptors in addition to the VEGF and KDR pathway. For example, some of the peptides are competitive inhibitors for integrin activation. Others affect interactions of endothelial cells with matrix components. Still others affect VEGF binding to KDR by binding the heparin sulfate moieties presented by endothelial cells.

The present invention provides peptides that target receptors and pathways which mediate several aspects of tumorigenesis like proliferation and invasion. For example, FGF4 is a potent oncogene (transforming gene) that is able to promote the uncontrolled growth of tumours. Increased PDGF-B production results in tumors with shortened latency, increased cellularity, regions of necrosis, and general high-grade character. MMP activation is strongly associated with tumor metastasis by permitting the movement of tumor cells through tissues (invasion).

In one embodiment of the invention, the peptides are bifunctional miniproteins capable of blocking the co-receptor activity of HS while at the same time blocking the binding of growth factors or other angiogenic ligands such as integrins. Blockage of the receptor can result in blocking multiple angiogenic pathways simultaneously, thereby achieving unexpected synergistic therapeutic activity.

The anti-angiogenic fusion peptide of the present invention comprises a first peptide linked to a second peptide through an optional linker peptide. The fusion peptides have inhibitory activity against one or more receptors involved in different angiogenic pathways. The fusion peptides are represented by the general formula (I):
(A)m−L−(B)n (I)

wherein L is an optional linker peptide comprising about 0 to about 10 amino acids;

wherein each A and B are independently peptides comprising about 1 to about 35 amino acids;

wherein m and n are independently integers from about 1 to about 3.

In certain embodiments the fusion peptide comprises a sequence wherein at least one of A and B comprises an amino sequence that binds one or more cell surface components such as VEGF receptors, integrin receptors, heparin, and FGF receptors. Preferred peptides of the present invention include but are not limited to the following peptide sequences:

| | | |
|---|---|---|
| ST100,032 | YDGRGDSVVYGLKKKAARGRRA | (SEQ ID NO.:1) |
| | ARGRR | |
| ST100,033 | PYAGRGDSVVYGLGGGPGAARG | (SEQ ID NO.:2) |
| | RRAARGRR | |
| ST100,061 | PYDGRGDSVVYGLRKKKAARGR | (SEQ ID NO.:3) |
| | RAARGRR | |
| ST100,062 | ATSLPPHSSQSPGGGPPAARGR | (SEQ ID NO.:4) |
| | RAARGRR | |
| ST100,063 | AARGRRAARGRRKKKAPYAGRG | (SEQ ID NO.:5) |
| | DSVVYGLR | |
| ST100,064 | RRGRAARRGRAAKKKRLGYVVS | (SEQ ID NO.:6) |
| | DGRGDYP | |
| ST100,065 | RLGYVVSDGRGDYPKKKRRGRA | (SEQ ID NO.:7) |
| | ARRGRAA | |
| ST100,066 | ATSLPPHSSQSPKKKAARGRRA | (SEQ ID NO.:8) |
| | ARGRR | |
| ST100,067 | PSQSSHPPLSTAKKKRRGRAAR | (SEQ ID NO.:9) |
| | RGRAA | |
| ST100,068 | RRGRAARRGRAAKKKPSQSSHP | (SEQ ID NO.:10) |
| | PLSTA | |
| ST100,072 | RRGRAAKKKRRGRAAKKKPSQS | (SEQ ID NO.:11) |
| | SHPPLSTA | |
| ST100,073 | RRGRAARRGRAARRGRAAKKKP | (SEQ ID NO.:12) |
| | SQSSHPPLSTA | |
| ST100,074 | RRGRAAKKKRRGRAAKKKRLGY | (SEQ ID NO.:13) |
| | VVSDGRGDYP | |
| ST100,075 | PSQSSHPPLSTAPPGGGPSQSS | (SEQ ID NO.:14) |
| | HPPLSTA | |
| ST100,076 | ATSLPPHSSQSPPPGGGPSQSS | (SEQ ID NO.:15) |
| | HPPLSTA | |
| ST100,077 | RLGYVVSDGRGDYP | (SEQ ID NO.:16) |
| ST100,078 | RRGRAARRGRAAKKK | (SEQ ID NO.:17) |
| ST100,079 | RAAKKRARAAKKRARAAKKRLG | (SEQ ID NO.:18) |
| | YVVSDGRGDYP | |
| ST100,080 | KRAAKKAAKRAKKAAKKAARLG | (SEQ ID NO.:19) |
| | YVVSDGRGDYP | |
| ST100,081 | RKKAARARKKAARARKKAARRL | (SEQ ID NO.:20) |
| | GYVVSDGRGDYP | |
| ST100,082 | RRGRAAKKKRRGRAAKKK | (SEQ ID NO.:21) |
| ST100,083 | RKRAARARKRAARARKRAARR | (SEQ ID NO.:22) |
| ST100,084 | RKRAARARKRAARARKRAARRL | (SEQ ID NO.:23) |
| | GYVVSDGRGDYP | |
| ST100,059 | PSQSSHPPLSTA | (SEQ ID NO.:30) |
| ST100,045 | ATSLPPHSSQSP | (SEQ ID NO.:31) |
| ST100,084 | ARRGRAARRGRAARRGRA | (SEQ ID NO.:68) |
| | (all D) | |
| ST100,085 | RRGRARRGRARRGRARRGKK | (SEQ ID NO.:69) |
| | (all D) | |
| ST100,086 | RKRAARARKRAARARKRAARRL | (SEQ ID NO.:70) |
| | GYVVSDGRGDYP (all D) | |
| ST100,087 | RKGRRKGRRKGRRKGRRKGKK | (SEQ ID NO.:71) |
| | (all D) | |
| ST100,088 | RRGRRRGRRRGRRRGKKRLGYV | (SEQ ID NO.:72) |
| | VSDGRGDYP (all D) | |
| ST100,089 | RRGRRRGRRRGRRRGKK | (SEQ ID NO.:73) |
| ST100,090 | RRQRRRGRRRQRRRGKK | (SEQ ID NO.:74) |
| ST100,091 | RRGRAAKKKRRGRAAKKKRLGY | (SEQ ID NO.:75) |
| | VVSDGRGDYP (all D) | |
| ST100,092 | RRARRRARRRARRRAKK | (SEQ ID NO.:76) |
| | (A are L-amino acids; | |
| | R are D amino acids) | |
| ST100,093 | RRGRRRGRRRGRRRGKKPSQSS | (SEQ ID NO.:77) |
| | HPPLSTA (all D) | |
| P deletion | RRGRRRGRRRGRRRGKKRLGYV | (SEQ ID NO.:78) |
| | VSDGRGDY | |
| RGD alpha | RRGRRRGRRRGRRRGKKRLGYV | (SEQ ID NO.:79) |
| mutation | VSDGRAAYP | |
| RGD beta | RRGRRRGRRRGRRRGKKRLGYV | (SEQ ID NO.:80) |
| mutation | VSAARGDYP | |
| RGD alpha | RRGRRRGRRRGRRRGKKRLGYV | (SEQ ID NO.:81) |
| and beta | VSAARAAYP | |
| mutation | | |
| SVV | RRGRRRGRRRGRRRGKKRAGAV | (SEQ ID NO.:82) |
| mutation | VSDGRGDYP | |
| Double | RRGRRRGRRRGRRRGKKRAGAV | (SEQ ID NO.:83) |
| mutant | VSAARAAP | |

The activity of the peptides SEQ ID NO.: 1 and SEQ ID NO.: 2 in blocking the binding of radiolabeled VEGF to endothelial cells is shown in FIG. 7.

Peptides of the invention may “comprise” the disclosed sequences, i.e., where the disclosed sequence is part of a larger peptide sequence that may or may not provide additional functional attributes to the disclosed peptide, such as enhanced solubility and/or stability, fusion to marker proteins for monitoring or measuring peptide activity or binding, larger peptides comprising immunogenic or antigenic peptides, etc. Preferred peptides of the invention may be described as including sequences “consisting essentially” of the disclosed sequences in addition to extraneous sequences which do not affect the anti-angiogenic activity and functional binding properties of the peptides. Alternatively, the peptides of the invention may consist only of the disclosed peptide sequences.

The sequences of the core peptides can be modified via conservative substitutions and/or by chemical modification or conjugation to other molecules in order to enhance parameters like solubility, serum stability, etc, while retaining anti-angiogenic activity and binding to KDR. In particular, the peptides of the invention may be acetylated at the N-terminus and/or amidated at the C-terminus, or conjugated, complexed or fused to molecules that enhance serum stability, including but not limited to albumin, immunoglobulins and fragments thereof, transferrin, lipoproteins, liposomes, α-2-macroglobulin and α-1-glycoprotein, polyethylene glycol and dextran. Such molecules are described in detail in U.S. Pat. No. 6,762,169, which is herein incorporated by reference in its entirety. Peptides and functional conservative variants having either L-amino acids or D-amino acids are included, particularly D-amino acid peptides having the reverse core sequences (retro inverso peptides), such as the peptide having amino acid sequence SEQ ID No. 30, shown above. Retro inverso peptides are suitable for pharmaceutical development because they are serum protease resistant, resulting in enhanced in vivo biological activity. In addition, the peptide may be modified by reducing one or more of the peptide bands to enhance stability (Pennington “solid-phase synthesis of peptides containing the CH2NH reduced band surrogate” in Molecular Biology, ed M. W. Pennington and B. M. Dunn 35(1994) 241-247 Humana Press Inc., Totowa, N.J.).

Conservative amino acid substitutions may be made with either naturally or non-naturally occurring amino acids. Appropriate conservative substitutions may be determined using any known scoring matrix or standard similarity comparison, including but not limited to the substitutions described in Bordo and Argos, Suggestions for ‘Safe’ Residue Substitutions in Site-Directed Mutagensis, J. Mol. Biol. 217(1991)721-729; Taylor, The Classification of Amino Acid Conservation, J. Theor. Biol. 119(1986)205-218; French and Robson, J. Mol. Evol. 19(1983)171; Pearson, Rapid and Sensitive Sequence Comparison with FASTP and FASTA, in Methods in Enzymology, ed. R. Doolittle (ISBN 0-12-182084-X, Academic Press, San Diego) 183 (1990) 63-98; and Johnson and Overington, 1993, J. Mol. Biol. 233: 716-738; and U.S. Pat. No. 5,994,125, each of which is herein incorporated by reference in its entirety. Some exemplary conservative substitutions based on a chemical property are included in Table 2 below.

TABLE 2
Exemplary Conservative Amino Acid Substitutions

| Interchangeable Amino Acids | Properties |
|---|---|
| Lysine (K), Arginine (R), | basic, large, polar, |
| Histidine (H), Ornithine, | hydrophilic, |
| Homoarginine | positively charged |
| Aspartic Acid (D), Glutamic | small, polar, acidic, |
| Acid (E), Asparagrine (N), | negatively charged |
| Glutamine (Q) | |
| Isoleucine (I), Leucine (L), | hydrophobic, large, |
| Methionine (M), Phenylalanine | polar or nonpolar |
| (F), Tryptophan (W), Tyrosine | |
| (Y), Valine (V), Cysteine (C), | |
| Noravaline, Homoalanine | |
| Alanine (A), Glycine (G), | small, nonpolar, |
| Serine (S), Threonine (T), | uncharged, hydrophilic |
| Cysteine (C), Asparagrine | |
| (N), Glutamine (Q), Homoalanine | |
| Phenylalanine (F), Tryptophan (W), | Aromatic |
| Tyrosine (Y), Histidine (H) | |
| Proline, Amino isobutyric acid | cyclic, bending |
| (Aib), Cycloleucine | |

The present invention also encompasses antibodies that specifically bind to the peptides disclosed herein. Exemplary antibodies include polyclonal, monoclonal, humanized, fully human, chimeric, bispecific, and heteroconjugate antibodies. Monoclonal antibodies may be prepared using hybridoma methods, such as those described by Kohler and Milstein, 1975, Nature 256: 495, which is herein incorporated by reference. Alternatively, lymphocytes may be immunized in vitro. The immunizing agent will typically include the peptide or a fusion protein thereof, further comprising a carrier or adjuvant protein.

Anti-idiotypic antibodies may also be prepared using standard procedures that exhibit properties substantially similar to the peptides as herein described. Such antibodies may therefore be used to inhibit or reduce VEGF-mediated stimulation of endothelial cells in the same manner as the disclosed peptides. Antibodies specific for the disclosed peptides may be labeled and used to detect the peptide, for instance in any of the receptor binding assays described herein. Alternatively, such antibodies may be used to purify recombinantly synthesized peptide.

Nucleic Acids

The present invention also encompasses isolated nucleic acids encoding the peptides described herein, as well as vectors comprising such nucleic acids for cloning (amplification of the DNA) or for expression. Various vectors are publicly available. The vector may, for example, be in the form of a plasmid, cosmid, viral particle, or phage. Such nucleic acids may be used to produce the peptide substrate, for instance by expressing the nucleic acid in a host cell. It will be understood by those skilled in the art that different nucleic acid sequences may encode the same amino acid sequence due to the degeneracy of the triplet code, and that the invention encompasses all possible nucleic acid sequences coding for the peptides described herein. Such nucleic acids may be synthetically prepared and cloned into any suitable vector using methods that are well known in the art.

Using well known cloning techniques, peptide coding sequences may be fused in frame to a signal sequence to allow secretion by the host cell. Alternatively, such peptides may be produced as a fusion to another protein, and thereafter separated and isolated by the use of a site specific protease. Such systems for producing peptides and proteins are commercially available. It will also be feasible to employ such host cells in methods for detecting expression of KDR by a test cell, or in methods of detecting VEGF activity in a sample, for instance by mixing a test cell or a sample with a host cell expressing a peptide of the invention and detecting binding of said host cell or said peptide or by detecting inhibition of VEGF activity. Suitable host cells include eukaryotic and prokaryotic cells. Vectors containing promoters for protein expression in specific host cells of interest are known and publicly available.

Nucleic acids and expression vectors encoding peptides of the invention may also be used in the therapeutic methods described herein, for instance as gene therapy vehicles to deliver the expressed peptide to the disease site. Suitable vectors are typically viral vectors, including DNA viruses, RNA viruses, and retroviruses (see Scanlon, 2004, Anticancer Res. 24(2A):501-4, for a recent review, which is herein incorporated by reference in its entirety). Controlled release systems, fabricated from natural and synthetic polymers, are also available for local delivery of vectors, which can avoid distribution to distant tissues, decrease toxicity to nontarget cells, and reduce the immune response to the vector (Pannier and Shea, 2004, Mol. Ther. 10(1): 19-26).

Methods of Use

The peptides of the present invention may be used in a variety of methods, including but not limited to methods of detecting KDR or other receptor expression and methods of detecting and/or inhibiting VEGF/receptor interaction and the interaction of other ligand/receptor pairs involved in angiogenesis as mentioned above. For instance, the peptides of the invention may be conjugated to radioactive or fluorescent imaging markers for the detection of KDR receptor expressing cells in vivo. Detection of aberrant or increased KDR expression could be an indication of ongoing disease, and could be used to localize of malignant tumors or diagnose eye diseases associated with excessive intraocular neovascularization.

The present invention also encompasses methods of using the peptides disclosed herein to screen for compounds that mimic the disclosed peptides (agonists) or prevent the effect of the peptides (antagonists). Screening assays for antagonist drug candidates are designed to identify compounds that bind to the KDR receptor, or otherwise interfere with the interaction of the disclosed peptides with KDR. Such screening assays will include assays amenable to high-throughput screening of chemical libraries, making them particularly suitable for identifying small molecule drug candidates. The assays can be performed in a variety of formats, including protein-protein binding assays, biochemical screening assays, immunoassays, and cell-based assays, which are well characterized in the art.

In particular, antagonists may be detected by combining a peptide of the invention and a potential antagonist with membrane-bound or surface-bound KDR receptors or recombinant receptors under appropriate conditions for a competitive inhibition assay. The peptide of the invention can be labeled, such as by radioactivity or fluorescence, such that the number of peptide molecules bound to the receptor can be used to determine the effectiveness of the potential antagonist.

The invention also encompasses methods for reducing VEGF-mediated angiogenesis, and for blocking VEGF binding to a KDR receptor or a KDR receptor peptide, comprising contacting a cell expressing kinase domain receptor (KDR) with the peptides described herein such that VEGF-mediated angiogenesis or VEGF binding, respectively, is reduced. In such methods, the KDR receptor or receptor peptide may be contacted with the peptide of the invention in the presence of VEGF or prior to being exposed to VEGF. Either the KDR or the peptide of the invention may be displayed on a synthetic surface, such as in a protein or peptide array. Alternatively, the KDR or KDR peptide may be expressed on the surface of a cell. KDR-expressing cells to be targeted by the methods of the invention can include either or both prokaryotic and eukaryotic cells. Such cells may be maintained in vitro, or they may be present in vivo, for instance in a patient or subject diagnosed with cancer or another angiogenesis-related disease.

The present invention also includes methods of treating a patient diagnosed with an angiogenesis-related disease with a therapeutically effective amount of any of peptides described herein, comprising administering said peptide to said patient such that said angiogenesis-related disease is reduced or inhibited. Exemplary angiogenesis-related diseases are described throughout this application, and include but are not limited to diseases selected from the group consisting of tumors and neoplasias, leukemia, multiple myeloma, hemangiomas, rheumatoid arthritis, atherosclerosis, idiopathic pulmonary fibrosis, vascular restenosis, arteriovenous malformations, meningioma, neovascular glaucoma, psoriasis, angiofibroma, hemophilic joints, hypertrophic scars, Osler-Weber syndrome, pyogenic granuloma retrolental fibroplasias, scleroderma, trachoma, vascular adhesion pathologies, synovitis, dermatitis, endometriosis, pterygium, diabetic retinopathy, neovascularization associated with corneal injury or grafts, wounds, sores, and ulcers (skin, gastric and duodenal).

In particular, the invention includes methods of treating a patient diagnosed with cancer with a therapeutically effective amount of any of the peptides described herein, comprising administering said peptide to said patient such that spread of said cancer is reduced or inhibited. Cancers treatable by the methods of the present invention include all solid tumor and metastatic cancers, including but not limited to those selected from the group consisting of kidney, colon, ovarian, prostate, pancreatic, lung, brain and skin cancers. Cancers such as neoplasias, leukemia and multiple myeloma can be treated with a therapeutically effective amount of the peptides described herein.

The present invention also includes methods of treating a patient diagnosed with a angiogenesis-associated eye disease with a therapeutically effective amount of any of the peptides described herein, comprising administering said peptide to said patient such that said eye disease is reduced or inhibited. Such eye diseases include any eye disease associated with abnormal intraocular neovascularization, including but not limited to retinopathy of prematurity, diabetic retinopathy, retinal vein occlusion, and macular degeneration.

The present invention also includes methods of treating a patient diagnosed with an angiogenesis-associated inflammatory condition with a therapeutically effective amount of any of the peptides described herein, comprising administering said peptide to said patient such that said inflammatory condition is reduced or inhibited. Such inflammatory conditions or diseases include any inflammatory disorder associated with expression of VEGF and activation of cells by VEGF, including but not limited to all types of arthritis and particularly rheumatoid arthritis and osteoarthritis, asthma, pulmonary fibrosis and dermatitis.

In another embodiment, the invention includes methods of treating a patient diagnosed with a heparin-sulfate mediated condition with a therapeutically effective amount of any of the peptides described herein. Heparin sulfate acts as co-receptors for a variety of ligands in physiological and pathological processes. For example, they mediate entry into the cells of pathogens like HIV and herpes simplex virus (HSV). Fusion proteins and miniproteins containing a heparin binding domain like those described in the this application can be used as therapeutic agents for the treatment of heparin-sulfate mediated disease or condition including but not limited to arterial and venous thrombosis, herpes simplex virus, African trypanosomiasis and onchocerciasis (River Blindness).

Pharmaceutical Formulations

For pharmaceutical uses, the compounds of the present invention may be used in combination with a pharmaceutically acceptable carrier, and can optionally include a pharmaceutically acceptable diluent or excipient. The present invention thus also provides pharmaceutical compositions suitable for administration to a subject. The carrier can be a liquid, so that the composition is adapted for parenteral administration, or can be solid, i.e., a tablet or pill formulated for oral administration. Further, the carrier can be in the form of a nebulizable liquid or solid so that the composition is adapted for inhalation. When administered parenterally, the composition should be pyrogen free and in an acceptable parenteral carrier. Active compounds can alternatively be formulated or encapsulated in liposomes, using known methods.

The pharmaceutical compositions of the invention comprise an effective amount of one or more peptides of the present invention in combination with the pharmaceutically acceptable carrier. The compositions may further comprise other known drugs suitable for the treatment of the particular disease being targeted. An effective amount of the compound of the present invention is that amount that blocks, inhibits or reduces VEGF stimulation of endothelial cells compared to that which would occur in the absence of the compound; in other words, an amount that decreases the angiogenic activity of the endothelium, compared to that which would occur in the absence of the compound. The effective amount (and the manner of administration) will be determined on an individual basis and will be based on the specific therapeutic molecule being used and a consideration of the subject (size, age, general health), the condition being treated (cancer, arthritis, eye disease, etc.), the severity of the symptoms to be treated, the result sought, the specific carrier or pharmaceutical formulation being used, the route of administration, and other factors as would be apparent to those skilled in the art. The effective amount can be determined by one of ordinary skill in the art using techniques as are known in the art. Therapeutically effective amounts of the compounds described herein can be determined using in vitro tests, animal models or other dose-response studies, as are known in the art.

The pharmaceutical compositions of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, and immunologically based formulations.

Liposomes are completely closed lipid bilayer membranes which contain entrapped aqueous volume. Liposomes are vesicles which may be unilamellar (single membrane) or multilamellar (onion-like structures characterized by multiple membrane bilayers, each separated from the next by an aqueous layer). The bilayer is composed of two lipid monolayers having a hydrophobic “tail” region and a hydrophilic “head” region. In the membrane bilayer, the hydrophobic (nonpolar) “tails” of the lipid monolayers orient toward the center of the bilayer, whereas the hydrophilic (polar) “heads” orient toward the aqueous phase.

The liposomes of the present invention may be formed by any of the methods known in the art. Several methods may be used to form the liposomes of the present invention. For example, multilamellar vesicles (MLVs), stable plurilamellar vesicles (SPLVs), small unilamellar vesicles (SUV), or reverse phase evaporation vesicles (REVs) may be used. Preferably, however, MLVs are extruded through filters forming large unilamellar vesicles (LUVs) of sizes dependent upon the filter size utilized. In general, polycarbonate filters of 30, 50, 60, 100, 200 or 800 nm pores may be used. In this method, disclosed in Cullis et al., U.S. Pat. No. 5,008,050, relevant portions of which are incorporated by reference herein, the liposome suspension may be repeatedly passed through the extrusion device resulting in a population of liposomes of homogeneous size distribution.

For example, the filtering may be performed through a straight-through membrane filter (a Nuclepore polycarbonate filter) or a tortuous path filter (e.g. a Nuclepore Membrafil filter (mixed cellulose esters) of 0.1 μm size), or by alternative size reduction techniques such as homogenization. The size of the liposomes may vary from about 0.03 to above about 2 microns in diameter; preferably about 0.05 to 0.3 microns and most preferably about 0.1 to about 0.2 microns. The size range includes liposomes that are MLVs, SPLVs, or LUVs.

Lipids which can be used in the liposome formulations of the present invention include synthetic or natural phospholipids and may include phosphatidylcholine (PC), phosphatidylethanolamine (PE), phosphatidylserine (PS), phosphatidylglycerol (PG), phosphatidic acid (PA), phosphatidylinositol (PI), sphingomyelin (SPM) and cardiolipin, among others, either alone or in combination, and also in combination with cholesterol. The phospholipids useful in the present invention may also include dimyristoylphosphatidylcholine (DMPC) and dimyristoylphosphatidylglycerol (DMPG). In other embodiments, distearoylphosphatidylcholine (DSPC), dipalmitoylphosphatidylcholine (DPPC), or hydrogenated soy phosphatidylcholine (HSPC) may also be used. Dimyristoylphosphatidylcholine (DMPC) and diarachidonoylphosphatidylcholine (DAPC) may similarly be used.

During preparation of the liposomes, organic solvents may also be used to suspend the lipids. Suitable organic solvents for use in the present invention include those with a variety of polarities and dielectric properties, which solubilize the lipids, for example, chloroform, methanol, ethanol, dimethylsulfoxide (DMSO), methylene chloride, and solvent mixtures such as benzene:methanol (70:30), among others. As a result, solutions (mixtures in which the lipids and other components are uniformly distributed throughout) containing the lipids are formed. Solvents are generally chosen on the basis of their biocompatability, low toxicity, and solubilization abilities.

To encapsulate the peptide(s) of the inventions into the liposomes, the methods described in Chakrabarti et al. U.S. Pat. No. 5,380,531, relevant portions of which are incorporated by reference, herein may be modified for use with the peptide(s) of the present invention.

Liposomes containing the amino acid and peptide formulations of the present invention may be used therapeutically in mammals, especially humans, in the treatment of a number of disease states or pharmacological conditions which require sustained release formulations as well as repeated administration. The mode of administration of the liposomes containing the agents of the present invention may determine the sites and cells in the organism to which the peptide may be delivered.

The liposomes of the present invention may be administered alone but will generally be administered in admixture with a pharmaceutical carrier selected with regard to the intended route of administration and standard pharmaceutical practice. The preparations may be injected parenterally, for example, intravenously. For parenteral administration, they can be used, for example, in the form of a sterile aqueous solution which may contain other solutes, for example, enough salts or glucose to make the solution isotonic, should isotonicity be necessary or desired. The liposomes of the present invention may also be employed subcutaneously or intramuscularly. Other uses, depending upon the particular properties of the preparation, may be envisioned by those skilled in the art.

For the oral mode of administration, the liposomal formulations of the present invention can be used in the form of tablets, capsules, lozenges, troches, powders, syrups, elixirs, aqueous solutions and suspensions, and the like. In the case of tablets, carriers which can be used include lactose, sodium citrate and salts of phosphoric acid. Various disintegrants such as starch, lubricating agents, and talc are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomal formulations of the present invention may be incorporated into dosage forms such as gels, oils, emulsions, and the like. These formulations may be administered by direct application as a cream, paste, ointment, gel, lotion or the like. For administration to humans in the treatment of disease states or pharmacological conditions, the prescribing physician will ultimately determine the appropriate dosage of the agent for a given human subject, and this can be expected to vary according to the age, weight and response of the individual as well as the pharmacokinetics of the agent used.

Also the nature and severity of the patient's disease state or condition will influence the dosage regimen. While it is expected that, in general, the dosage of the drug in liposomal form will be about that employed for the free drug, in some cases, it may be necessary to administer dosages outside these limits.

The pharmaceutical compositions of the invention further comprise a depot formulation of biopolymers such as biodegradable microspheres. Biodegradable microspheres are used to control drug release rates and to target drugs to specific sites in the body, thereby optimizing their therapeutic response, decreasing toxic side effects, and eliminating the inconvenience of repeated injections. Biodegradable microspheres have the advantage over large polymer implants in that they do not require surgical procedures for implantation and removal.

The biodegradable microspheres used in the context of the invention are formedb with a polymer which delays the release of the peptides and maintains, at the site of action, a therapeutically effective concentration for a prolonged period of time.

The polymer can be chosen from ethylcellulose, polystyrene, poly(ε-caprolactone), poly(lactic acid) and poly(lactic acid-co-glycolic acid) (PLGA). PLGA copolymer is one of the synthetic biodegradable and biocompatible polymers that has reproducible and slow-release characteristics. An advantage of PLGA copolymers is that their degradation rate ranges from months to years and is a function of the polymer molecular weight and the ratio of polylactic acid to polyglycolic acid residues. Several products using PLGA for parenteral applications are currently on the market, including Lupron Depot and Zoladex in the United States and Enantone Depot, Decapeptil, and Pariodel LA in Europe (see Yonsei, Med J. 2000 December; 41(6):720-34 for review).

The pharmaceutical compositions of the invention may further be prepared, packaged, or sold in a formulation suitable for nasal administration as increased permeability has been shown through the tight junction of the nasal epithelialium (Pietro and Woolley, The Science behind Nastech's intranasal drug delivery technology. Manufacturing Chemist, August, 2003). Such formulations may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for nasal delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

The compounds of the present invention can be administered acutely (i.e., during the onset or shortly after events leading to inflammation), or can be administered during the course of a degenerative disease to reduce or ameliorate the progression of symptoms that would otherwise occur. The timing and interval of administration is varied according to the subject's symptoms, and can be administered at an interval of several hours to several days, over a time course of hours, days, weeks or longer, as would be determined by one skilled in the art. A typical daily regime can be from about 0.01 μg/kg body weight per day, from about 1 mg/kg body weight per day, from about 10 mg/kg body weight per day, from about 100 mg/kg body weight per day.

The compounds of the invention may be administered intravenously (IV), orally, intranasally, intraocularly, intramuscularly (IM), intrathecally, or by any suitable route in view of the peptide, the peptide formulation and the disease to be treated. Peptides for the treatment of inflammatory arthritis can be injected directly into the synovial fluid. Peptides for the treatment of solid tumors may be injected directly into the tumor. Peptides for the treatment of skin diseases may be applied topically, for instance in the form of a lotion or spray. Intrathecal administration, i.e. for the treatment of brain tumors, can comprise injection directly into the brain. Alternatively, peptides may be coupled or conjugated to a second molecule (a “carrier”), which is a peptide or non-proteinaceous moiety selected for its ability to penetrate the blood-brain barrier and transport the active agent across the blood-brain barrier. Examples of suitable carriers are disclosed in U.S. Pat. Nos. 4,902,505; 5,604,198; and 5,017,566, which are herein incorporated by reference in their entirety.

An alternative method of administering peptides of the present invention is carried out by administering to the subject a vector carrying a nucleic acid sequence encoding the peptide, where the vector is capable of directing expression and secretion of the peptide. Suitable vectors are typically viral vectors, including DNA viruses, RNA viruses, and retroviruses. Techniques for utilizing vector delivery systems and carrying out gene therapy are known in the art (see Lundstrom, 2003, Trends Biotechnol. 21(3):117-22, for a recent review).

The following examples are provided to describe and illustrate the present invention. As such, they should not be construed to limit the scope of the invention. Those in the art will well appreciate that many other embodiments also fall within the scope of the invention, as it is described herein above and in the claims The following examples are provided to describe and illustrate the present invention. As such, they should not be construed to limit the scope of the invention. Those in the art will well appreciate that many other embodiments also fall within the scope of the invention, as it is described herein above and in the claims.

### EXAMPLES

### Example 1

### Identification of Novel Human VEGF Receptor KDR Binding Peptides by Minicell Panning

Methods

A minicell display library comprising random 30-mer oligonucleotides genetically fused to the gene encoding the 17K antigen of Rickettsia rickettsii in the vector pBS (Bluescript) was constructed essentially as described in U.S. patent application 20030105310,;which is herein incorporated by reference in its entirety. The library was transformed into E. coli DS410, and transformed cells were grown in a 250 mL culture overnight in rich medium (Terrific Broth). Minicells were purified by differential centrifugation at 9.3 K rpm.

An ELISA-based binding assay for minicell screening was performed as follows:

Costar high binding plate 3361 was coated with 5 μg/ml KDR receptor (R&D systems, 357-KD) diluted with 100 mM sodium bicarbonate 30 mM sodium carbonate pH 9.5 coating buffer—50 μl/well. Coating buffer was added alone to two wells as negative control wells.

Plate was incubated at 4° C. over-weekend with slight rotation.

Next morning: Minicell random library aliquot (10% of pellet) was resuspended in 1 ml PBS. 1 μl Bodipy was added and minicells were stained 10 min while rotating at room temperature. The sample was spun 1 min at 13000 rpm and the pellet was washed 3×5 min with 900 μl PBS with rotation at room temperature. The sample was spun 1 min at 13000 rpm and the pellet resuspended in 560 μl PBS for assay.

Unbound KDR was removed from high binding plate to new plate to save.

The plate washed once briefly with 200 μl PBS.

Labeled minicells added: the minicells were diluted 1:1 with appropriate PBS buffer prepared 2× concentration of eventual wash condition (i.e., PBS, PBS with 500 mM NaCl, PBS with 1M NaCl, PBS+0.2% NP-40, PBS+0.02% SDS) and loaded 50 μl/well with 0.1% BSA and 25 μg/ml kanamycin. Minicells were added to control wells as well.

The plate was sealed and incubated 4° C. overnight as above (total incubation=18 hrs).

Unbound minicells were removed to a new plate to save.

The plate was washed 3×1 min with 200 μl of appropriate buffer—PBS, PBS with 250 mM NaCl, PBS with 500 mM NaCl, PBS+0.1% NP-40, PBS+0.01% SDS. 50 μl PBS/well was added and plate was incubated three hours at 4° C.

Plate was viewed under microscope at 20× and 40× magnification for labeled minicells.

Minicell DNA was extracted from positive wells via phenol-chloroform and transformed into competent DH5alpha cells.

Colonies were isolated and cultured in 5 mL LB+100 μg/ml Amp overnight at 37° C.

DNA was miniprepped from 1.5 mL of culture via Qiagen method and submitted to Keck facility for sequencing.

Sequences were compared to literature for sequences having significant homology.

Homology Analysis

Six clones were obtained and their sequences were compared to sequences disclosed in the following two papers:

Binetruy-Tournaire R. et al., 2000, Identification of a peptide blocking vascular endothelial growth factor (VEGF)-mediated angiogenesis, EMBO J. 19(7):1525-33.

Lu D. et al., 2003, Tailoring in vitro selection for a picomolar affinity human antibody directed against vascular endothelial growth factor receptor 2 for enhanced neutralizing activity, J. Biol. Chem. 278(44):43496-507.

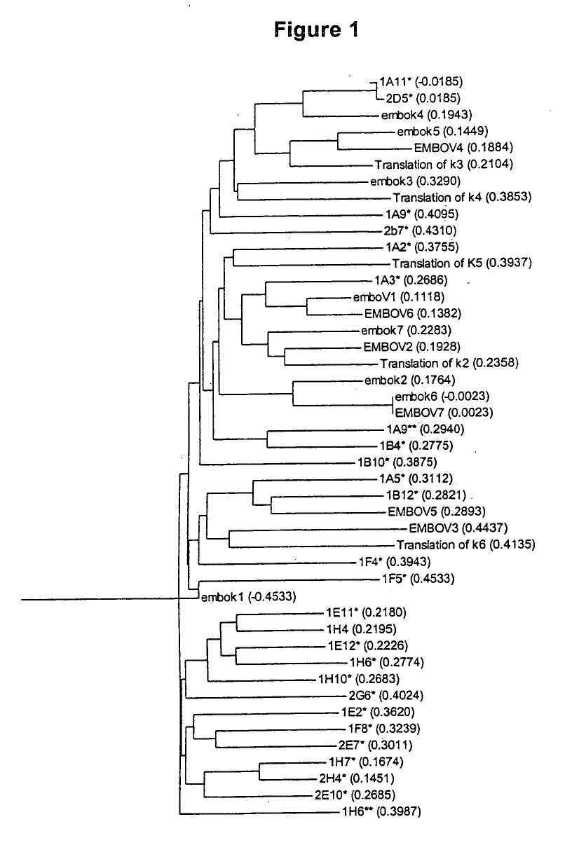

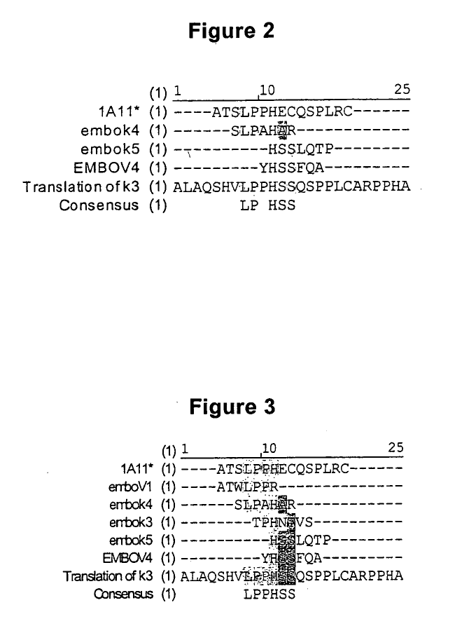

Binetruy-Tournaire et al. used immobilized KDR to screen a phage display library. Lu et al. used phage display library to further define the fine binding specificities of two fully human neutralizing KDR-specific antibodies. As shown in FIG. 1, by comparing the clones identified by minicell display screening with the peptides disclosed in the two papers referenced above, a series of subgroups were identified (see FIG. 1, a phylogenetic tree generated by clustalW using Vector NTI). Of particular interest is the subgroup at the top of the alignment tree, comprising the peptides: EmboK4 (SEQ ID No. 32), EmboK5 (SEQ ID No. 33) and EmboV4 (SEQ ID No. 34) from the paper by Binetruy-Tournaire et al., the two peptides 1A11 and 2D5 (which have the same sequence (SEQ ID No. 35) and therefore will be considered as one) from the paper by Lu et al., and the clone K3 (SEQ ID No. 36) obtained by minicell display technology. The alignment of these peptides is shown in FIG. 2.

The high level of sequence homology between the peptide sequences in FIG. 2 suggested that the K3 peptide or partial fragments of this peptide would have anti-angiogenic properties. Further homology searching with the sequence of this peptide revealed another pocket of homology between K3 and the two peptides disclosed by Binetruy-Tournaire et al., EmboV1 (SEQ ID No. 37) and EmboK3 (SEQ ID No. 38). The final alignment of all of these peptides is shown in FIG. 3. This alignment revealed the existence of a consensus sequence that is highly conserved among all the peptides, LPPHSS. While Binetruy-Tournaire et al. discussed the relevance of the LPP sequence for biological activity and mentioned the presence of the HSS sequence in two of the isolated peptides, the combination of both these subsequences together in a single peptide is not disclosed. Nevertheless, in view of the alignment of the sequences and the comparison to the K3 peptide identified using minicell display technology, the present inventors predicted that a peptide with the sequence LPPHSS would have anti-angiogenic properties substantially different and more useful than either of the two isolated sequences by themselves.

In addition, the homology alignment revealed two further regions of consensus. The region ATS that is present in the amino terminal portion of the peptide 1A11 is partially conserved in the EmboV1 (see FIG. 2). Further, the serine residue is present in alignment in EmboK4. Accordingly, the present inventors also predicted that this region would contribute anti-angiogenic properties, and that a peptide with the sequence ATSLPPHSS would have anti-angiogenic properties substantially different and more useful than either of the three isolated sequences alone. The other region of homology covers the subsequence QSP, present in the C-terminal region of peptide 1A11 and in the peptide K3. In addition, the serine is conserved in the peptide EmboK3. Accordingly, the present inventors also predicted that this region would contribute anti-angiogenic properties, and that a peptide with the sequence ATSLPPHSSQSP (ST100,038; SEQ ID NO.: 29) would have anti-angiogenic properties substantially different and more useful than any of the four isolated sequences alone.

### Example 2

### Generation and Studies of D-Amino Acid Derivatives in 1% or 10% Serum

L-amino acid peptides are unstable when exposed to serum due to their susceptibility to serum protease digestion. It was hypothesized that generating serum stable derivatives of L-amino acid peptides would improve their pharmaceutical attributes. For this reason D-amino acid derivatives of the original peptides were generated and tested for serum stability.

Methods

A stock solution of 1 mM peptide dissolved in water was made. The stock was then diluted to 100 μM in either OptiMem media+100 μl/ml penicillin/100 μg/ml streptomycin sulfate+1% fetal calf serum or in OptiMem+Pen/Strep+10% serum. The diluted samples were placed in a 24 well tissue culture plate in an incubator. Aliquots of 50-100 μl were removed at 4, 6, 18, 24, 48 and 72 hrs and frozen at −70° C. until analysis.

Samples of 20 μl were separated on a C18 column (4.8×250 mm) with a gradient of acetonitrile/water 0.1% TFA and analyzed using a single quad mass spectrometer. Singly or multiply charged peaks were detected depending on the mass of the peptide. Peptide degradation was determined in two ways: loss of peak area in the chromatogram produced using the mass spectrometer as the detector and loss of the main peak in the mass spectrum with simultaneous appearance of a peak(s) from a breakdown product.

Serum stability of L-amino acid peptides

1% serum: 48 hours

10% serum: <24 hours

Complete serum: 15 minutes

Serum stability of D-amino acid peptides

Complete serum: >24 hours

The results of the analysis as summarized above show that L-amino acid peptides are much less stable than D-aminoacid peptides in higher amount of serum, 10% or complete serum, due to their susceptibility to protease digestion.

Experiments were then performed to determine whether replacing L-amino acid peptides with D-amino acid peptides resulted in active and stable peptides. D-amino acid peptides can be made by generating a D-amino acid peptide with the same sequence as a L-amino acid peptide or by preparing a retro inverso form of a peptide. ST100,045 (SEQ ID NO.: 31) has the same sequence as ST100,038 (SEQ ID NO.: 29) was tested against ST100,059 (SEQ ID NO.: 30) which is the retro inverso version of ST100,038 and a control. Only the retro inverso form of ST100,038, (ST100,059; SEQ ID NO.: 30) was found to be biologically active.

Derivatives of the peptides described in this application can incorporate a direct replaced, a complete reverse, and/or middle rotated reversed version of one or more of the disclosed domains. For example, the D-amino acid derivatives of the miniprotein ST100,061 (SEQ ID NO.: 3), named ST100,064 (SEQ ID NO.: 6) and ST100,065 (SEQ ID NO.: 7) were generated. ST100,064 (SEQ ID NO.: 6) is the direct inversion of ST100,061 (SEQ ID NO.: 3) and is much more active both in its ability to bind heparin (see Example 3) and its ability to induce tumor cell death (see Example 5) than the middle rotated replaced version ST100,065 (SEQ ID NO.: 7).

### Example 3

### Characterization of Heparin Binding Activity of Bifunctional Peptides In Vitro

Methods

The following peptides were synthesized to test for anti-angiogenic activities in vitro and in vivo:

| | | |
|---|---|---|
| ST100,032 | YDGRGDSVVYGLKKKAARGRRA | (SEQ ID NO.:1) |
| | ARGRR | |
| ST100,033 | PYAGRGDSVVYGLGGGPGAARG | (SEQ ID NO.:2) |
| | RRAARGRR | |
| ST100,061 | PYDGRGDSVVYGLRKKKAARGR | (SEQ ID NO.:3) |
| | RAARGRR | |
| ST100,062 | ATSLPPHSSQSPGGGPPAARGR | (SEQ ID NO.:4) |
| | RAARGRR | |
| ST100,063 | AARGRRAARGRRKKKAPYAGRG | (SEQ ID NO.:5) |
| | DSVVYGLR | |
| ST100,066 | ATSLPPHSSQSPKKKAARGRRA | (SEQ ID NO.:8) |
| | ARGRR | |

In addition, the following variants of ST100,064 (SEQ ID NO.: 6) and ST100,065 (SEQ ID NO.: 7) were synthesized using D-amino acids as opposed to L-amino acids to test the effect of the modification on activity and serum stability:

| | | |
|---|---|---|
| ST100,064 | RRGRAARRGRAAKKKRLGYVVS | (SEQ ID NO.:6) |
| | DGRGDYP | |
| ST100,065 | RLGYVVSDGRGDYPKKKRRGRA | (SEQ ID NO.:7) |
| | ARRGRAA | |
| ST100,067 | PSQSSHPPLSTAKKKRRGRAAR | (SEQ ID NO.:9) |
| | RGRAA | |
| ST100,068 | RRGRAARRGRAAKKKPSQSSHP | (SEQ ID NO.:10) |
| | PLSTA | |
| ST100,072 | RRGRAAKKKRRGRAAKKKPSQS | (SEQ ID NO.:11) |
| | SHPPLSTA | |
| ST100,073 | RRGRAARRGRAARRGRAAKKKP | (SEQ ID NO.:12) |
| | SQSSHPPLSTA | |
| ST100,074 | RRGRAAKKKRRGRAAKKKRLGY | (SEQ ID NO.:13) |
| | VVSDGRGDYP | |
| ST100,075 | PSQSSHPPLSTAPPGGGPSQSS | (SEQ ID NO.:14) |
| | HPPLSTA | |
| ST100,076 | ATSLPPHSSQSPPPGGGPSQSS | (SEQ ID NO.:15) |
| | HPPLSTA | |
| ST100,077 | RLGYVVSDGRGDYP | (SEQ ID NO.:16) |
| ST100,078 | RRGRAARRGRAAKKK | (SEQ ID NO.:17) |
| ST100,079 | RAAKKRARAAKKRARAAKKRLG | (SEQ ID NO.:18) |
| | YVVSDGRGDYP | |
| ST100,080 | KRAAKKAAKRAKKAAKKAARLG | (SEQ ID NO.:19) |
| | YVVSDGRGDYP | |
| ST100,081 | RKKAARARKKAARARKKAARRL | (SEQ ID NO.:20) |
| | GYVVSDGRGDYP | |
| ST100,082 | RRGRAAKKKRRGRAAKKK | (SEQ ID NO.:21) |
| ST100,083 | RKRAARARKRAARARKRAARR | (SEQ ID NO.:22) |
| ST100,084 | RKRAARARKRAARARKRAARRL | (SEQ ID NO.:23) |
| | GYVVSDGRGDYP | |
| ST100,086 | RRGRARRGRARRGRARRGKK | (SEQ ID NO.:28) |

Liquid chromatography was used to determine the relative levels of heparin binding activity of the individual heparin binding domains and of the anti-angiogenic miniproteins that contains them. In this assay, the strength of the heparin binding activity is proportional to the amount of NaCl that is required to elute the peptide bound to the heparin column. Peptides with low binding activity are eluted with lower NaCl concentration, whereas higher concentrations of NaCl are required for peptides with higher binding activity.

Hi Trap Heparin HP column (1 ml, Amersham Biosciences) was equilibrated with 10 column volumes (CV) of equilibration (EQ) buffer=10 mM NaH2PO4 pH 7. All buffers were loaded onto columns via syringes. 500 μl fractions are collected (flow rate=1 ml/minute). 500 μg of peptide (1 mg/ml, resuspended in EQ buffer) was added to each column and the flow through was collected for analysis. The columns were then washed with 3 CV of EQ buffer. Peptides are then eluted with a step gradient of 500, 625, 750, 875 mM NaCl in EQ buffer, 2 CV per each step. A final step of 3 CV of 1000 mM NaCl in EQ buffer was collected in 500 μl fractions. The A210 nm was measured using EQ and elution buffers as blanks.

Results

As reported in Table 3, the activity of individual heparin binding domains depends on the number of basic residues and their organization. It was found that peptides with a greater number of basic residues have a higher binding activity. Domain bbbxxbx (SEQ ID NO.: 84) was found to bind stronger to NaCl that the domain bbxbxx (SEQ ID NO.: 85). ST100,059 (SEQ ID NO.: 30) which has no heparin binding domain, elutes at 0 mM NaCl. Peptides ST100,064 (SEQ ID NO.: 6) and ST100,065 (SEQ ID NO.: 7) which contain the domain bbxbxx (SEQ ID NO.: 85) were found to bind less strongly than ST100,082 (SEQ ID NO.: 21) which contains the domain bbbxxbx (SEQ ID NO.: 84).

Further refinement of the heparin binding domain led to the generation of a set of peptides, ST100,087 (SEQ ID NO.: 71), ST100,089 (SEQ ID NO.: 73), and ST100,090 (SEQ ID NO.: 74), that showed an even greater increase in heparin binding, as indicated by the increased molarity of NaCl required for elution. This stronger ability to bind heparin resulted in a greater biological potency as shown in the following examples.

These sets of peptides show a very high affinity for heparin, as indicated by the very high molarity of NaCl that is required for elution. Other heparin binding motif containing proteins with anti-angiogenic activities have much lower affinity, requiring about 350 mM NaCl for elution (see Sasaki et al., 1990, EMBO J. 18(22): 6240-8 and Chen et al., 2001, J Biol Chem. 276(2): 1276-84). These peptides therefore represent improvements to the previous art. In addition, they have much higher affinity for heparin than angiogenic growth factors like FGFs have for cellular heparan sulfate, indicating that they are able to work as effective competitors of these growth factors.

TABLE 3

| | [NaCl] for | fraction |
|---|---|---|
| | elution | number |
| ST100,059 | 0 mM | |
| ST100,064 | 875 mM | |
| ST100,065 | 750 mM | |
| ST100,068 | 875 mM | |
| ST100,072 | 1000 mM | 26-27 |
| ST100,073 | 1000 mM | 26-27 |
| ST100,074 | 1000 mM | 27-28 |
| ST100,078 | 875 mM | |
| ST100,079 | 1000 mM | |
| ST100,081 | 1000 mM | 26-27 |
| ST100,082 | 1000 mM | 26-27 |
| ST100,083 | 1100 mM | |
| ST100,085 | 1100 mM | 31-32 |
| ST100,087 | 1500 mM | 35-36 |
| ST100,089 | 1300 M | 34-35 |
| ST100,090 | 1300 mM | 33-34 |

### Example 4

### Characterization of Anti-Angiogenic Activity of Bifunctional Peptides In Vitro

Methods

The anti-angiogenic activities of the peptides were tested by measuring the level of inhibition of VEGF and bFGF mediated survival/proliferation of Bovine Retinal Endothelial Cells (BRE), Human Dermal Microvasculature Endothelial Cells, and Human Umbilical Vein Endothelial Cells, all of which are standard cell lines used to test anti-angiogenic compounds.

Bovine retinal endothelial (BRE) cells were maintained in Cambrex EG2 media. For non-adherent cell assays, on day one cells were starved for either 6 hours or overnight, then trypsinized and plated in 96-well plates in 100 μl of Optimem plus 1% fetal bovine serum (FBS). One hundred μl of Optimem plus 1% FBS was added to the wells containing, where appropriate, VEGF to a final concentration of 25 ng/ml, and the various peptides to final concentrations as described. For adherent cells, cells were plated in 96-well plates in complete media, allowed to adhere overnight, washed in starvation media (Optimem plus 1% FBS) and then starved during the day. At the end of the day, 100 μl of Optimem plus 1% FBS was added to the wells containing, where appropriate, VEGF to a final concentration of 25 ng/ml and the various peptides to final concentrations as described.

Human umbilical cord endothelial (HUVEC) cells were maintained in Cambrex EGM-2MV media. On day one, cells were starved overnight in 1% FBS in M200 media (Cascade Biologicals). The morning after, the media were replaced with serum-free media (control) or media containing 25 ng/ml of human VEGF165 and the various peptides to final concentrations as described.

In all cases, after 72 hours incubation, the amount of live cells in each well was measured with the WST1 assay (Roche).

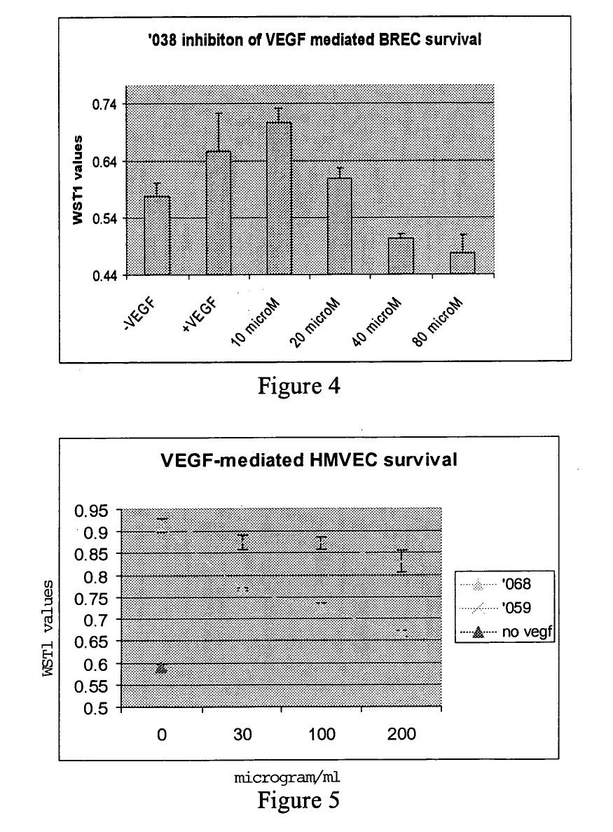

FIG. 4 is a bar graph showing how increasing concentrations of peptide ST100,038 (SEQ ID NO.: 29) caused the amount of WST-1 to decrease and therefore the number of live cells to decrease. Student's t-test analysis of the data reveals that these decreases are statistically significant. Concentrations above 40 μM completely abolished the statistically significant VEGF-induced increase in WST-1 value and actually resulted in even lower values than observed in cells without VEGF stimulation. The most likely explanation is that the peptide inhibits the stimulation of the cells by the growth factors (VEGF) present in the media.

FIG. 5 illustrates the inhibition of VEGF activation by two of the synthesized peptides. VEGF stimulation was inhibited with increasing doses of peptides ST100,059 (SEQ ID NO.: 30) and ST100,068 (SEQ ID NO.: 10). ST100,059 is the retro inverso form of ST100,038 (SEQ ID NO.: 29), whereas ST100,068 is a miniprotein obtained by fusing ST100,059 to an heparin binding domain. As show by the graph, ST100,068 was found to be more potent in blocking VEGF stimulation because of the VEGF co-receptor activity of heparan sulfate.

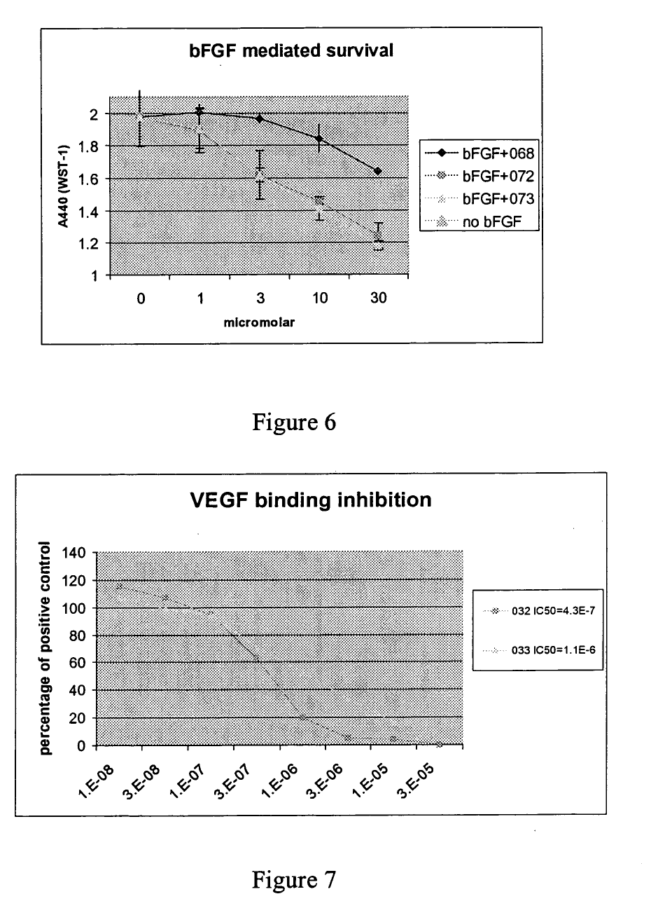

FIG. 6 illustrates the inhibition of bFGF activation by two derivatives of ST100,068 (SEQ ID NO.: 10). ST100,072 (SEQ ID NO.: 11) and ST100,073 (SEQ ID NO.: 12) are miniproteins obtained by replacing the heparin binding domain of ST100,068 with more potent heparin binding domains. As illustrated by the graph, they are more potent in blocking bFGF stimulation confirming that better heparin binding activity confers more potent anti-angiogenic activity.

FIG. 7 illustrates the inhibition of VEGF binding to its receptor by two miniproteins wherein a heparin binding domain is linked to an integrin binding domain. VEGF binding was inhibited with increasing doses of peptides ST100,032 (SEQ ID NO.: 1) and ST100,033 (SEQ ID NO.: 2). Both peptides achieved an almost 100% inhibition at a concentration of 30 μM. The IC50 values for peptides ST100,032 and ST100,033 are 430 nM and 1.1 μM, respectively. This result suggests that the synthetic peptides are capable of disrupting the binding of VEGF to its receptor even if they are only blocking the co-receptor activity mediated by HS.

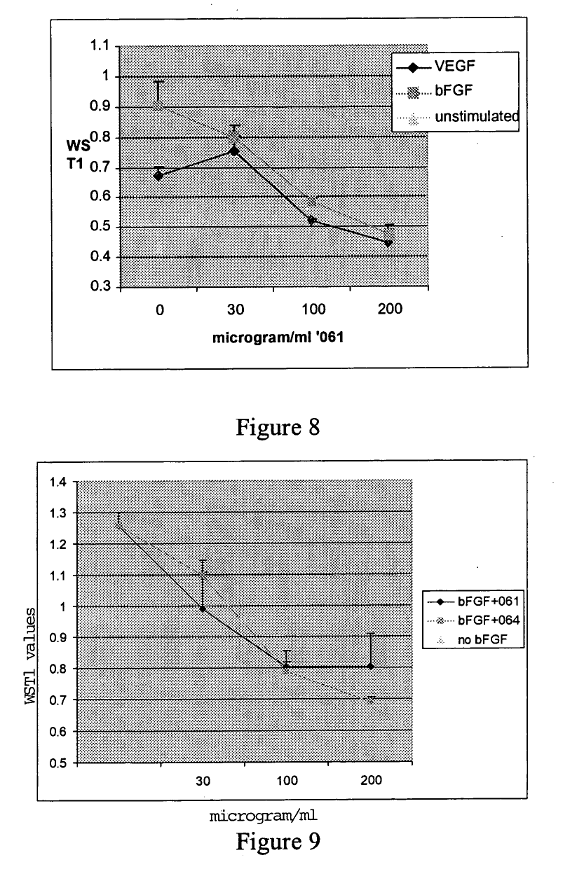

In a further experiment, the anti-angiogenic activity of peptide ST100,061 (SEQ ID NO.: 3), a derivative of ST100,032 (SEQ-ID NO.: 1), at concentrations of 30, 100, and 200 μg/ml was tested by measuring the level of inhibition of VEGF and bFGF mediated survival/proliferation in human dermal microvasculature endothelial cells. FIG. 8 shows that increasing concentrations of peptide ST100,061 decreased the amount of WST-1 and therefore the number of live cells. The decrease in the amount of WST-1 in both the VEGF and bFGF mediated survival of endothelial cells was comparable, showing that the peptide is effective in inhibiting both VEGF and bFGF.

The activity of ST100,061 (SEQ ID NO.: 3) in inhibiting bFGF mediated survival was then compared to its retro-inverso form ST100,064 (SEQ ID NO.: 6), in human umbilical vein endothelial cells. FIG. 9 indicates that ST100,064 can inhibit bFGF mediated survival as effectively as ST100,061.

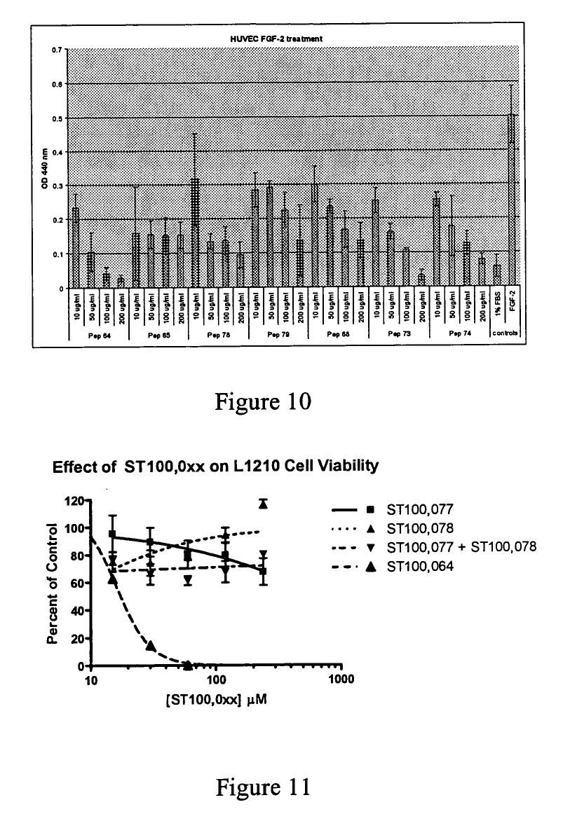

The ability of several peptides to inhibit bFGF mediated survival of human umbilical vein endothelial cells was then compared. FIG. 10 indicates that those miniproteins with strong heparin binding domains like ST100,064, ST100,073 and ST100,074 are the most active in inhibiting bFGF stimulation. ST100,078, which encodes for the heparin binding domain, by itself is not as potent. The data show that the linking of the heparin binding domain to either the KDR binding domain or the integrin binding domain results in synergistic anti-angiogenic activity.

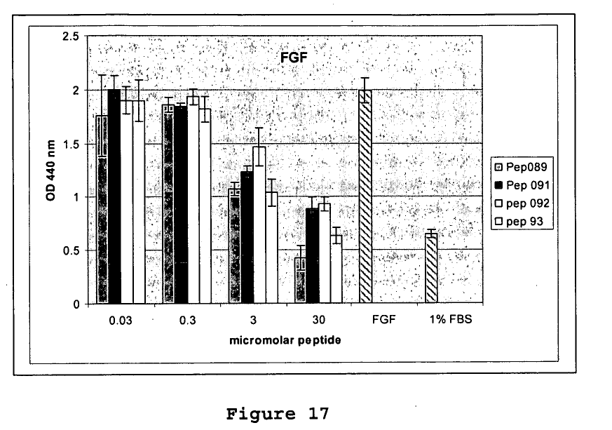

Even stronger heparin binding domains were then tested for their ability to inhibit bFGF mediated survival of human umbilical vein endothelial cells. FIG. 17 indicates that these stronger heparin binding domains are the most active in inhibiting bFGF stimulation. For instance, ST100,093 (SEQ ID NO.: 77), which encodes for a stronger heparin binding domain linked to the KDR binding domain (as opposed to the integrin binding domain) is more potent than similar peptides with weaker heparin binding domains.

### Example 5

### Characterization of Anti-Proliferative Activity of Miniprotein In Vitro Against Tumor Cells

Methods

Peptides to be tested were prepared at a stock concentration of 10 mM in sterile phosphate buffered saline. Cancer cell lines obtained from the American Type Culture Collection (MG-63, HT1080, A498, BxPC3, 786-0, PC-3, B16F1, B16F10, P388D1, Jurkat, MOLT4, THP-1, U-937, L1210, RPMI 8226, NCI H929, U266B1, K562) were cultured under appropriate conditions as described in the literature. Cell culture media and reagents were obtained from ATCC (Manassas, Va.), Invitrogen (Carlsbad, Calif.) or Mediatech (Herndon, Va.). Exponentially growing cultures were used for cell proliferation assays. Adherent cells were plated at a concentration of 100000 cells per milliter in growth media overnight (18-24 h) and treated the next day in a low serum media (growth media with 1% FBS for MG-63, HT1080, A498, BxPC3, PC-3, B16F1, B16F10. 786-0 cells were treated in media with 5% FBS). Suspension cell lines (P388D1, Jurkat, MOLT4, THP-1, U-937, L1210, RPMI 8226, NCI H929, U266B1, and K562) were diluted to a concentration of 100,000 cells per ml and treated on the same day with peptides. Peptides were diluted in treatment media and cells were treated for 48 or 72 hours depending on the cell line. Each dose was tested in triplicate for each experiment, and experiments were repeated for a minimum of three discrete times. After incubation, the relative number of cells was determined using WST-1 (Roche Applied Science). A 9.5 μl aliquot of WST-1 was added to each well. The plate was immediately read at 440 nm using a Bio-Tek PowerWave XS microplate reader, incubated for 2-3 hours at 37° C. and then read again. Cell proliferation was determined as the percent of the control cell proliferation. The absorbance of each well at time 0 was subtracted from the value of the final reading. Afterwards the blank values were averaged and subtracted from each test and control value. Finally, each test absorbance was divided by the average of the control absorbances and multiplied by 100 to obtain the percent of control.

To determine the EC₅₀ for each peptide the percent of control growth was plotted versus the log of the drug concentration and fitted using Prism software (GraphPad Software Inc) to the sigmoidal dose response equation.

Results

In addition to endothelial cells, many other cell lineages, including tumor cells, require integrin activation for proper cellular homeostasis. A set of tumor cells were treated with miniproteins containing the integrin binding domain to test whether these miniproteins were able to block proliferation or induces cell death. As shown in the graph of FIG. 11, peptide ST100,064 (SEQ ID NO.: 6) containing a heparin binding domain and an integrin binding domain was able to block cell proliferation and induce cell death. Neither ST100,077 (SEQ ID NO.: 16) encoding the integrin binding domain or ST100,078 (SEQ ID NO.: 17) encoding the heparin binding domain or the simple combination of the two peptides (ST100,077 and ST100,078) blocked cell proliferation or induced cell death. The results demonstrate the synergistic ability of a protein of the invention to kill tumor cells.

Table 4 reports the IC50 for the set of tumor cells treated with 4 different miniproteins containing an integrin binding domain linked to a heparin binding domain. The general point is that the greater is the ability to bind heparin, as previously defined, the greater the potency, as indicated by a lower IC50.

TABLE 4

| | Organ/Cancer | | | | | |
|---|---|---|---|---|---|---|
| CELL LINE | derivative | TYPE | ST100,065 | ST100,064 | ST100,074 | ST100,088 |
| HT1080 | fibrosarcoma | Adherent | R | 48.8 ± 5 | 10.11 ± 0.2 | |
| A498 | kidney | Adherent | R | R | 39.62 ± 7.1 | |
| | carcinoma | | | | | |
| BxPC3 | pancreatic | Adherent | 117.08 ± 23.31 | 57.07 ± 10.29 | 25.03 ± 2.11 | |
| | adenocarcinoma | | | | | |
| MG-63 | osteosarcoma | Adherent | R | 20.14 ± 1.07 | 8.92 ± 1.24 | 4.67 ± 1.67 |
| 786-0 | kidney | Adherent | 114.4 ± 32 | 23.5 ± 3.3 | 9.7 ± 1.09 | |
| | adenocarcinoma | | | | | |
| PC-3 | prostate | Adherent | 246.9 ± 69.9 | 138.25 ± 36.3 | 96.7 ± 29.3 | |
| | adenocarcinoma | | | | | |
| MDAMB453 | breast | Adherent | 251.5 ± 129.4 | 67.33 ± 8.19 | 28.3 ± 5.24 | 3.36 ± 0.94 |
| | metastatic | | | | | |
| | carcinoma | | | | | |
| P388D1 | mouse leukemia | Suspension | R | 22.9 ± 4.54 | 9.18 ± 2.38 | |
| Jurkat | T cell leukemia | Suspension | 173.8 ± 65.9 | 62.3 ± 8.5 | 36.89 ± 2.18 | |
| MOLT4 | lymphoblastic | Suspension | R | 42.7 ± 3.9 | 24.05 ± 0.27 | |
| | leukemia | | | | | |
| THP-1 | monocytic | Suspension | R | 19.24 ± 2.4 | 13.11 ± 0.27 | |
| | leukemia | | | | | |
| U-937 | histiocytic | Suspension | 220.5 ± 45.8 | 65.36 ± 20.7 | 36.89 ± 6.28 | |
| | lymphoma | | | | | |
| L1210 | mouse myeloma | Suspension | 39 ± 5 | 18 ± 5 | 9 ± 1 | 3 |
| RPMI 8226 | myeloma | Suspension | 327 | 40 ± 5 | 42 ± 4 | 12 |
| NCI H929 | myeloma; | Suspension | 143 | 25 ± 7 | 22 ± 6 | 9 |
| | plasmacytoma | | | | | |
| U266B1 | myeloma; | Suspension | ND | 182 ± 30 | 63 ± 5 | 21 |
| | plasmacytoma | | | | | |
| K-562 | chronic | Suspension | R | R | 39.8 ± 5.8 | |
| | myelogenous | | | | | |
| | leukemia | | | | | |

### Example 6

### Characterization of Anti-tumor Activity of KDR Binding Peptides and Miniprotein In Vivo in the Subcutaneous B16 Melanoma Tumor

Because of the importance of the angiogenic process for tumorigenesis, miniproteins as described herein, were hypothesized to show good anti-tumor activity. The peptides of the invention were tested in an in vivo model of anti-tumor activity. This model compares the growth of subcutaneous B16 melanoma tumor in vivo either untreated or treated with various amount of miniproteins described in this application. This model is widely accepted in the art as a model to test the anti-tumor activity of compounds that inhibit tumor growth because they have anti-angiogenic activity.

Methods

Male C57BL/6 mice were obtained with a mean body weight of 20±2 g. Mouse B16-F1 melanoma cells were implanted subcutaneously (5×105 cell per animal). Peptides (formulated in water) were administered ip daily at the amount indicated starting the day after cells injection. In general, tumors became palpable around 9 days after injection of cells. Tumor were then measured every 2 days.

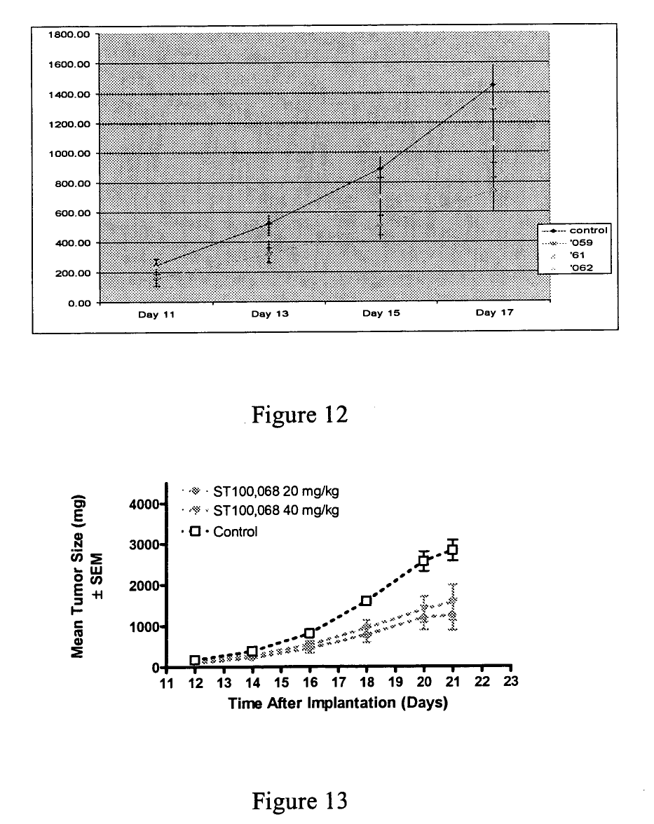

The quantitative results of the first experiment are presented in FIG. 12. The graph shows that ST100,059 (SEQ ID NO.: 30) and ST100,062 (SEQ ID NO.: 4) peptides clearly inhibit tumor growth, with ST100,059 being statistically significant in an ANOVA analysis P<0.05, while ST100,062 has P>0.05. ST100,061 (SEQ ID NO.: 3) may be less active due to being quickly degraded in serum in an inactive form cleaved in the heparin binding domain.

FIG. 13 is a graph comparing inhibition of growth of melanoma B16 tumor implanted subcutaneously and treated in vivo with 20 mg/kg and 40 mg/kg daily IP of ST100,068 (SEQ ID NO.: 10) as compared to untreated controls. This experiment shows that the ST100,059 (SEQ ID NO.: 30) derivative ST100,068 is able to inhibit tumor growth.

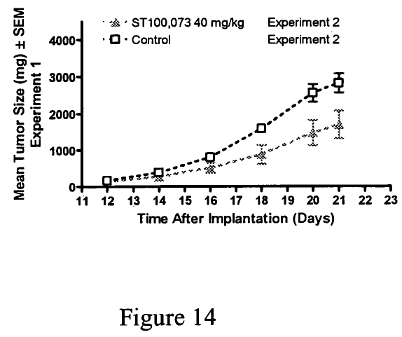

FIG. 14 is a graph comparing inhibition of growth of melanoma B16 tumor implanted subcutaneously and treated in vivo with 20 mg/kg daily IP of ST100,073 (SEQ ID NO.: 12) as compared to untreated controls. This experiment shows that the ST100,068 derivative ST100,073 is able to inhibit tumor growth.

### Example 7

### Characterization of Anti-Tumor Activity of Miniproteins In Vivo in the L1210 Murine Leukemia Intravenous Model

Because the miniproteins containing a heparin binding domain linked to an integrin binding domain showed the ability to induce cell death in addition to having anti-angiogenesis properties, it was hypothesized that these miniproteins should demonstrate anti-tumor activity in models where tumorigenesis does not require angiogenesis. Therefore, the peptides of the invention were tested in an in vivo model where L1210 murine leukemia are implanted intravenously. In this model, the tumor cell proliferate directly in the bloodstream and do not require angiogenesis. This model is widely accepted in the art as a model to test the anti-tumor activity of a compound to induce cell death.

Methods

Antitumor activity of test peptides, administered intraperitoneally (IP), were evaluated against L1210 murine leukemia cells implanted intravenously (IV) in DBA/2 mice. This cell line was chosen because all of the compounds showed good in vitro anti-tumor activity against it.

Studies generally consisted of randomly-assigned groups of 8 mice per group, which were inoculated IV with 1×10⁵ cells per mouse from an in vivo leukemia cell line. In addition to groups tested with test peptides, studies usually included a vehicle-treated control group and a positive control group treated with an agent known to be active in the L1210 leukemia model. Starting one day after tumor inoculation (inoculation day defined as Day 0), mice were treated IP with either vehicle or test peptides in various schedules. Generally this consisted of treatment every other day for approximately 1 week (e.g., Days 1, 3, 5 and 7). A positive agent (e.g., cyclophosphamide) was usually given as a single IP injection on Day 1. All dosing solutions were prepared on each day of treatment. Survival was monitored daily and body weights were measured twice weekly. Anti-tumor activity was assessed by the increase in lifespan of the treated groups in comparison to the vehicle-treated control group. Studies with the L1210 leukemia model were limited to 30 days.

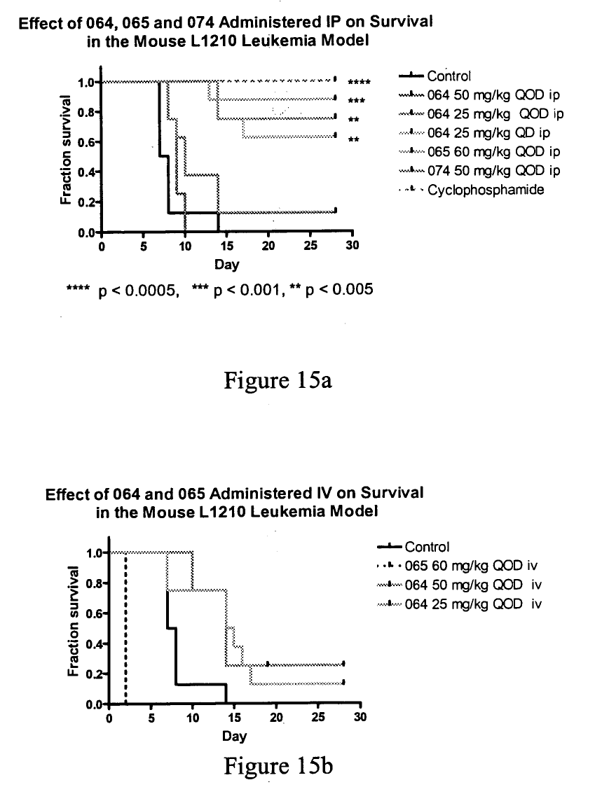

As the graph in FIG. 15a illustrates, ST100,064 (SEQ ID NO.: 6), ST100,065 (SEQ ID NO.: 7) and ST100,074 (SEQ ID NO.: 13) when dosed IP resulted in statistically significant increases in survival. Sixty to eighty percent of the mice (cured mice) survived longer than 30 days. The graph in FIG. 15b shows that the peptides demonstrated a reduced activity when injected IV, most likely due to their quick excretion from the bloodstream. For this reason, pharmaceutical composition of these peptides that increase the circulating halftime by methods commonly known in the art should result in improved efficacy.

### Example 8

### Characterization of Anti-Tumor Activity of Miniproteins In Vivo in the RPMI-8226 Human Myeloma Subcutaneous Xenograft Model

Methods

Anti-tumor efficacy of test peptides was evaluated against RPMI-8226 human myeloma xenografts implanted subcutaneously (sc) in severe compromised immunodeficient (scid) mice.

Studies generally consisted of randomly-assigned groups of 8 or 10 mice per group, which were implanted sc with myeloma fragments (30-40 mg). In addition to groups tested with test peptides, studies usually included a vehicle-treated control group and a positive control group treated with an agent known to be active in the RPMI-8226 model. In one type of schedule, mice were treated IP with either vehicle or test peptides starting one day after tumor implantation (implantation day defined as Day 0). Test peptides and vehicle were generally administered IP daily for 3-4 weeks.

Dosing solutions of the test peptides were prepared weekly and kept at −20° C. between injections. All agents were administered on the basis of individual animal body weights (e.g., 0.1 ml/10 g body weight). Mice were observed daily for survival. Each tumor was measured by caliper in two dimensions and converted to tumor mass using the formula for a prolate ellipsoid (a X b²/2), where a is the longer dimension and b is the smaller dimension, and assuming unit density (1 mm³=1 mg). Tumor measurements were recorded twice weekly. Body weights were also recorded twice weekly. Anti-tumor activity was assessed by the delay in tumor growth of the treated groups in comparison to the vehicle-treated control group, partial and complete regressions, and tumor-free survivors. The studies were limited to 60 days.

Results

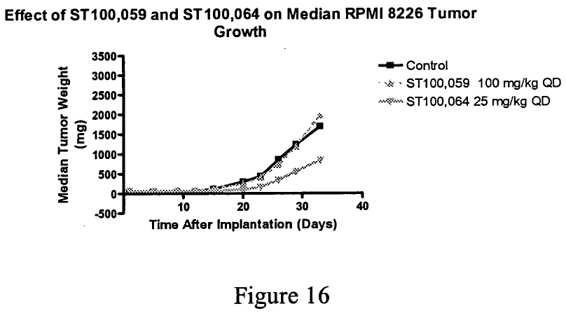

FIG. 16 is a graph comparing inhibition of growth of RPMI-8226 human myeloma xenografts in vivo treated with 25 mg/kg daily IP of ST100,064 or 100 mg/kg daily IP of ST100,059 (SEQ ID NO.: 30) as compared to untreated controls. This experiment shows that the ST100,064 (SEQ ID NO.: 6) peptide, which acts directly by inducing tumor cell death, is able to inhibit tumor growth while ST100,059, which only acts by inhibiting angiogenesis, does not inhibit tumor growth.

All publications, patents and patent applications discussed herein are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed:

1. An anti-angiogenic fusion peptide comprising a first peptide linked to a second peptide through an optional linker peptide.

2. The fusion peptide of claim 1, wherein the linker peptide is present between the first and second peptide.

3. The fusion peptide of claim 1, wherein the first peptide and the second peptide have inhibitory activity against one or more receptors involved in different angiogenic pathways.

4. The fusion peptide of claim 1, wherein said fusion peptide is represented by the general formula:
(A)m−L−(B)n
wherein L is an optional linker peptide comprising about 0-35 amino acids;
wherein each A and B are independently peptides comprising about 1-about 35 amino acids;
wherein m and n are independently integers from about 1-3.

5. The fusion peptide of claim 4, wherein A and B have inhibitory activity against one or more receptors involved in different angiogenic pathways.

6. The fusion peptide of claim 4, wherein at least one of A and B comprises an amino sequence that binds one or more cell surface components.

7. The fusion peptide of claim 6, wherein said cell surface components are selected from the group consisting of a VEGF receptor, an integrin receptor, heparin, and a FGF receptor.

8. The fusion peptide of claim 7, wherein said VEGF receptor is a tyrosine kinase receptor selected from the group consisting of VEGFRI (Flt-1) and VEGFRII (KDR).

9. The fusion peptide of claim 6, wherein A comprises an amino sequence that binds one or more cell surface components.

10. The fusion peptide of claim 9, wherein said amino acid sequence binds a VEGF receptor.

11. The fusion peptide of claim 10, wherein said amino sequence binds Flt-1.

12. The fusion peptide of claim 10, wherein said amino sequence binds KDR.

13. The fusion peptide of claim 12 wherein said binding inhibits VEGF binding to KDR.

14. The fusion peptide of claim 9, wherein said amino sequence binds an integrin receptor.

15. The fusion peptide of claim 14, wherein said binding inhibits the binding of ligands to integrin receptors.

16. The fusion peptide of claim 6, wherein B comprises an amino sequence that binds one or more cell surface components.

17. The fusion peptide of claim 16, wherein said amino sequence binds heparin.

18. The fusion peptide of claim 4, wherein said peptide comprises L-amino acids.

19. The fusion peptide of claim 4, wherein said peptide comprise D-amino acids.

20. The fusion peptide of claim 19, comprising a retro-inverso isomer.

21. The fusion peptide of claim 20, wherein the retro-inverso isomer comprises about 2-70 D-amino acids.

22. The fusion peptide of claim 1 comprising the amino acid sequence YDGRGDSVVYGLKKKAARGRRAARGRR (SEQ ID NO.: 1) or conservative substitutions thereof.

23. The fusion peptide of claim 22, wherein said peptide comprises L-amino acids.

24. The fusion peptide of claim 1 comprising the amino acid sequence PYAGRGDSVVYGLGGGPGAARGRRAARGRR (SEQ ID NO.: 2) or conservative substitutions thereof.

25. The fusion peptide of claim 24, wherein said peptide comprises L-amino acids.

26. The fusion peptide of claim 1 comprising the amino acid sequence PYDGRGDSVVYGLRKKKAARGRRAARGRR (SEQ ID NO.: 3) or conservative substitutions thereof.

27. The fusion peptide of claim 26, wherein said peptide comprises L-amino acids.

28. The fusion peptide of claim 1 comprising the amino acid sequence ATSLPPHSSQSP (SEQ ID NO.: 29) or conservative substitutions thereof.

29. The fusion peptide of claim 28, wherein said peptide comprises L-amino acids.

30. The fusion peptide of claim 1 comprising the amino acid sequence AARGRRAARGRRKKKAPYAGRGDSVVYGLR (SEQ ID NO.: 5) or conservative substitutions thereof.

31. The fusion peptide of claim 30, wherein said peptide comprises L-amino acids.

32. The fusion peptide of claim 1 comprising the amino acid sequence RRGRAARRGRAAKKKRLGYVVSDGRGDYP (SEQ ID NO.: 6) or conservative substitutions thereof.

33. The fusion peptide of claim 32, wherein said peptide comprises D-amino acids.

34. The fusion peptide of claim 1 comprising the amino acid sequence RLGYVVSDGRGDYPKKKRRGRAARRGRAA (SEQ ID NO.: 7) or conservative substitutions thereof.

35. The fusion peptide of claim 34, wherein said peptide comprises D-amino acids.

36. The fusion peptide of claim 1, wherein the peptide is amino-terminally modified.

37. The fusion peptide of claim 36, wherein said peptide comprises an acetylated amino terminus.

38. The fusion peptide of claim 1, wherein the peptide is carboxy-terminally modified.

39. The fusion peptide of claim 38, wherein said peptide comprises an amidated carboxy terminal.

40. The fusion peptide of claim 1, wherein said peptide is conjugated to a moiety that enhances serum stability.

41. The fusion peptide of claim 40, wherein said moiety is selected from the group consisting of albumin, immunoglobulins and fragments thereof, transferrin, lipoproteins, liposomes, α-2-macroglobulin and α-1-glycoprotein, polyethelene glycol and dextran.

42. The fusion peptide of claim 1, wherein one or more peptide bonds are reduced.

43. The fusion peptide of claim 1, wherein the peptide contains a heparin binding domain.

44. The fusion peptide of claim 43, wherein said heparin binding domain consists of (bbxbxx) (SEQ ID NO.: 85) or (bbbxxbx) (SEQ ID NO.: 84), wherein
each B is independently selected from the group consisting of arginine and lysine residues and
each X is independently any amino acid residue.

45. A pharmaceutical composition comprising the peptide of claim 1.

46. The composition of claim 45 further comprising a pharmaceutically acceptable carrier.

47. A method for reducing angiogenesis, comprising contacting a cell with a peptide of claim 1.

48. A method for blocking VEGF binding to a KDR receptor or a KDR receptor peptide and integrin binding to an integrin receptor, comprising contacting said KDR receptor, said KDR receptor peptide, or said integrin receptor with the peptide of claim 1 such that VEGF binding and endothelial cell activation are blocked or inhibited.

49. The method of claim 48, wherein said KDR receptor, KDR receptor peptide, or integrin receptor is expressed on the surface of a cell.

50. The method of claim 48, wherein said cell is maintained in vitro.

51. The method of claim 48, wherein said cell is in vivo.

52. The method of claim 48, wherein said cell is in a subject diagnosed with cancer.

53. The method of claim 48, wherein said KDR receptor, KDR receptor peptide, or integrin receptor is displayed in a peptide array on a surface.

54. A method of treating a patient diagnosed with cancer with a therapeutically effective amount of a peptide of claim 1, comprising administering said peptide to said patient such that the growth or spread of said cancer is reduced or inhibited.

55. The method of claim 54, wherein said cancer is a solid tumor cancer selected from the group consisting of kidney, colon, ovarian, prostate, pancreatic, lung, brain, breast and skin.

56. A method of treating a patient diagnosed with a angiogenesis-associated eye disease with a therapeutically effective amount of a peptide of claim 1, comprising administering said peptide to said patient such that said eye disease is reduced or inhibited.

57. The method of claim 56, wherein said eye disease is selected from the group
consisting of retinopathy of prematurity, diabetic retinopathy, retinal vein occlusion, macular degeneration and neovascularization associated with corneal injury or grafts.

58. A method of treating a patient diagnosed with an angiogenesis-related disease with a therapeutically effective amount of a peptide of claim 1, comprising administering said peptide to said patient such that said angiogenesis-related disease is reduced or inhibited.

59. The method of claim 58, wherein said angiogenesis-related disease is selected from the group consisting of leukemia, multiple myeloma, hemangiomas, rheumatoid arthritis, atherosclerosis, idiopathic pulmonary fibrosis, vascular restenosis, arteriovenous malformations, meningiomas, neovascular glaucoma, psoriasis, angiofibroma, hemophilic joints, hypertrophic scars, Osler-Weber syndrome, pyogenic granuloma, retrolental fibroplasias, scleroderma, trachoma, vascular adhesion pathologines, synovitis, dermatitis, endometriosis, pterygium, wounds, sores, and ulcers (skin, gastric and duodenal).

60. The method of claim 54, wherein said cancer is a hematological malignancy.

61. The method of claim 60, wherein said hematological malignancy is leukemia or multiple myeloma.

62. The fusion peptide of claim 1 comprising an amino acid sequence corresponding to SEQ ID NO.: 1 through SEQ ID NO.: 31, SEQ ID NO.: 67 through SEQ ID NO.: 85 or conservative substitutions thereof.

63. The fusion peptide of claim 1, wherein the first peptide is a heparin binding domain and the second peptide is an integrin binding domain.

64. The fusion peptide of claim 1, wherein the fusion peptide is a miniprotein.
